(19) United States
(12) Patent Application Publication
(10) Pub. No.: US 2023/0241376 A1
(43) Pub. Date: Aug. 3, 2023

(54) Paddle Electrode Assembly

(71) Applicant: Saluda Medical Pty Ltd, Artarmon (AU)

(72) Inventors: Peter Scott Vallack Single, Artarmon (AU); John Louis Parker, Artarmon (AU); Zubin Zarir Nanavati, Artarmon (AU)

(73) Assignee: Saluda Medical Pty Ltd, Artarmon (AU)

(21) Appl. No.: 18/163,214

(22) Filed: Feb. 1, 2023

(30) Foreign Application Priority Data
Feb. 2, 2022 (AU) 2022900196

(51) Int. Cl. A61N 1/05; A61N 1/04; A61N 1/36

(52) U.S. Cl. CPC A61N 1/0553; A61N 1/0416; A61N 1/36125

(57) ABSTRACT

An implantable device for controllably stimulating a neural target. The device configured to electrically couple to a paddle electrode assembly, the paddle electrode assembly comprising a plurality of electrodes including a first group of one or more electrodes arranged on a ventral surface of a paddle body, and a second group of one or more electrodes arranged on a dorsal surface of the paddle body. The implantable device comprises stimulation circuitry, configured to provide stimulation energy to one or more electrodes of the paddle electrode assembly, measurement circuitry, configured to measure a response evoked from the neural target by the stimulation energy and sensed by one or more electrodes of the paddle electrode assembly, and an electrode selection module. The electrode selection module is configured to select at least one first electrode from the plurality of electrodes of the paddle electrode assembly and electrically couple the first electrode to the stimulation circuitry, and select at least one second electrode from the plurality of electrodes of the paddle electrode assembly and electrically couple the second electrode to the measurement circuitry.

### CROSS REFERENCE TO RELATED APPLICATIONS

The current application claims the benefit of and priority to Australian Patent Application No. 2022900196 filed Feb. 2, 2022. The disclosure of Australian Patent Application No. 2022900196 is here by incorporated by reference in its entirety for all purposes.

### TECHNICAL FIELD

The present invention relates to providing stimulus to generate a neural response, and in particular relates to the use of a paddle electrode assembly to provide stimulus and measure a neural response to stimulus.

### BACKGROUND OF THE INVENTION

There are a range of situations in which it is desirable to apply neural stimuli in order to alter neural function, a process known as neuromodulation. For example, neuromodulation is used to treat a variety of disorders including chronic neuropathic pain, Parkinson's disease, and migraine. A neuromodulation system applies an electrical pulse (stimulus) to neural tissue (fibres, or neurons) in order to generate a therapeutic effect. In general, the electrical stimulus generated by a neuromodulation system evokes a neural action potential in a neural fibre which then has either an inhibitory or excitatory effect. Inhibitory effects can be used to modulate an undesired process such as the transmission of pain, or excitatory effects may be used to cause a desired effect such as the contraction of a muscle.

When used to relieve neuropathic pain originating in the trunk and limbs, the electrical pulse is applied to the dorsal column (DC) of the spinal cord, a procedure referred to as spinal cord stimulation (SCS). Such a system typically comprises an implanted electrical pulse generator, and a power source such as a battery that may be transcutaneously rechargeable by wireless means, such as inductive transfer. An electrode array is connected to the pulse generator, and is positioned adjacent the target neural fibre(s) in the spinal cord, typically in the dorsal epidural space above the dorsal column. An electrical pulse of sufficient intensity applied to the target neural fibres by a stimulus electrode causes the depolarisation of neurons in the fibres, which in turn generates a response known as an action potential in the fibres. Action potentials propagate along the fibres in orthodromic (towards the head, or rostral) and antidromic (towards the cauda, or caudal) directions. The fibres being stimulated in this way inhibit the transmission of pain from a region of the body innervated by the target neural fibres (the dermatome) to the brain. To sustain the pain relief effects, stimuli are applied repeatedly, for example at a frequency in the range of 30 Hz-100 Hz.

For effective and comfortable neuromodulation, it is necessary to maintain stimulus intensity above a recruitment threshold. Stimuli below the recruitment threshold will fail to recruit sufficient neurons to generate action potentials with a therapeutic effect. In almost all neuromodulation applications, response from a single class of fibre is desired, but the stimulus waveforms employed can evoke action potentials in other classes of fibres which cause unwanted side effects. In pain relief, is therefore necessary to apply stimuli with intensity below a comfort threshold, above which uncomfortable or painful percepts arise due to over-recruitment of Aβ fibres. When recruitment is too large, Aβ fibres produce uncomfortable sensations. Stimulation at high intensity may even recruit Aδ fibres, which are sensory nerve fibres associated with acute pain, cold and pressure sensation. It is therefore desirable to maintain stimulus intensity within a therapeutic range between the recruitment threshold and the comfort threshold.

The task of maintaining appropriate neural recruitment is made more difficult by electrode migration (change in position over time) and/or postural changes of the implant recipient (patient), either of which can significantly alter the neural recruitment arising from a given stimulus, and therefore the therapeutic range. There is room in the epidural space for the electrode array to move, and such array movement from migration or posture change alters the electrode-to-fibre distance and thus the recruitment efficacy of a given stimulus. Moreover, the spinal cord itself can move within the cerebrospinal fluid (CSF) with respect to the dura. During postural changes, the amount of CSF and/or the distance between the spinal cord and the electrode can change significantly. This effect is so large that postural changes alone can cause a previously comfortable and effective stimulus regime to become either ineffectual or painful.

Another control problem facing neuromodulation systems of all types is achieving neural recruitment at a sufficient level for therapeutic effect, but at minimal expenditure of energy. The power consumption of the stimulation paradigm has a direct effect on battery requirements which in turn affects the device's physical size and lifetime. For rechargeable systems, increased power consumption results in more frequent charging and, given that batteries only permit a limited number of charging cycles, ultimately this reduces the implanted lifetime of the device.

Attempts have been made to address such problems by way of feedback or closed-loop control, such as using the methods set forth in International Patent Publication No. WO2012155188 by the present applicant. Feedback control seeks to compensate for relative nerve/electrode movement by controlling the intensity of the delivered stimuli so as to maintain a substantially constant neural recruitment. The intensity of a neural response evoked by a stimulus may be used as a feedback variable representative of the amount of neural recruitment. A signal representative of the neural response may be generated by a measurement electrode in electrical communication with the recruited neural fibres, and processed to obtain the feedback variable. Based on the response intensity, the intensity of the applied stimulus may be adjusted to maintain the response within a therapeutic range.

It is therefore desirable to accurately detect and record a neural response evoked by the stimulus. The action potentials generated by the depolarisation of a large number of fibres by a stimulus sum to form a measurable signal known as an evoked compound action potential (ECAP). Accordingly, an ECAP is the sum of responses from a large number of single fibre action potentials. The ECAP generated from the depolarisation of a group of similar fibres may be measured at a measurement electrode as a positive peak potential, then a negative peak, followed by a second positive peak. This morphology is caused by the region of activation passing the measurement electrode as the action potentials propagate along the individual fibres.

Approaches proposed for obtaining a neural measurement are described by the present applicant in International Patent Publication No. WO2012/155183, the contents of which are incorporated herein by reference.

The accuracy of a neural response measurement can be impacted by artefacts that are generated as a result of neural stimulation. In practice, many non-ideal aspects of a circuit lead to the generation of undesirable artefacts. Some artefacts manifest due to the physical characteristics of the electrode array. Some artefacts manifest due to how the electrodes of the electrode array are configured and utilised to enable neural stimulation and neural response measurement. An artefact may manifest as a positive potential, a negative potential, or a changing potential that combines with the potential generated by the neural target in response to the neural stimulation. The artefact potential shifts the measured ECAP response from the actual ECAP response generated by the neural target, resulting in the measured ECAP response not accurately representing the actual ECAP response.

Therefore, a need exists for a solution to ameliorate the effect of artefacts that may generate in response to stimulation of a neural target.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

In this specification, a statement that an element may be “at least one of” a list of options is to be understood to mean that the element may be any one of the listed options, or may be any combination of two or more of the listed options.

### SUMMARY OF THE INVENTION

According to an aspect of the present technology, there is provided an implantable device for controllably stimulating a neural target. The device is configured to electrically couple to a paddle electrode assembly. The paddle electrode assembly comprising a plurality of electrodes arranged on a paddle body. The plurality of electrodes includes a first group of one or more electrodes arranged on a ventral surface of the paddle body, and a second group of one or more electrodes arranged on a dorsal surface of the paddle body. The implantable device comprises stimulation circuitry, configured to provide stimulation energy to one or more electrodes of the paddle electrode assembly, measurement circuitry, configured to measure a response evoked from the neural target by the stimulation energy and sensed by one or more electrodes of the paddle electrode assembly, and an electrode selection module. The electrode selection module is configured to select at least one first electrode from the plurality of electrodes of the paddle electrode assembly and electrically couple the first electrode to the stimulation circuitry, and select at least one second electrode from the plurality of electrodes of the paddle electrode assembly and electrically couple the second electrode to the measurement circuitry.

In some implementations, the second group of one or more electrodes arranged on the dorsal surface of the paddle body comprises the at least one second electrode. In some implementations, the second group of one or more electrodes includes at least two electrodes.

In some implementations, the electrode selection module is configured to select the at least one second electrode from the plurality of electrodes of the paddle electrode assembly based on the position of the at least one second electrode relative to the at least one first electrode. In some implementations, the electrode selection module is configured to select the at least one first electrode based on the position of the at least one first electrode relative to the neural target. In some implementations, the electrode selection module is configured to select the at least one first electrode based on a measured response evoked from the neural target.

In some implementations, the at least one first electrode comprises a cathode electrode, configured to sink stimulus current, and two anode electrodes, configured to source return currents. The cathode electrode may be located in a position in the paddle electrode assembly between the two anode electrodes.

In some implementations, the at least one second electrode comprises a recording electrode, and a reference electrode. The measurement circuitry may be configured to determine a different in potential between the recording electrode and the reference electrode.

In some implementations, the electrode selection module is configured to select the at least one second electrode from the plurality of electrodes of the paddle electrode assembly to maximise a distance between the at least one first electrode and the at least one second electrode.

In some implementations, the implantable device further comprises the paddle electrode assembly. The paddle electrode assembly may comprise a lead body having a proximal end and a distal end, and a paddle disposed at the distal end of the lead body. The paddle may comprise a paddle body, comprising a ventral surface and a dorsal surface, and a plurality of electrodes arranged on the ventral surface of the paddle body. The lead body may be configured to electrically couple each electrode of the plurality of electrodes to the stimulation circuitry of the implantable stimulation device, and the lead body may be configured to electrically couple each electrode of the plurality of electrodes to the measurement circuitry of the implantable stimulation device.

According to another aspect of the present technology, there is provided a method of stimulating a neural target by an implanted paddle electrode assembly. The paddle electrode assembly comprises a plurality of electrodes arranged on a paddle body. The method comprises selecting, from the plurality of electrodes, a first subset of electrodes, selecting, from the plurality of electrodes, a second subset of electrodes, providing stimulation energy to the first subset of electrodes to stimulate the neural target, and measuring a response of the neural target, the response evoked from the neural target by the stimulation energy, from the second subset of electrodes, wherein the plurality of electrodes includes a first group of one or more electrodes arranged on a ventral surface of the paddle body, and a second group of one or more electrodes arranged on a dorsal surface of the paddle body.

In some implementations, the second group of one or more electrodes arranged on a dorsal surface of the paddle board comprises the second subset of electrodes. In some implementations, the second group of electrodes includes at least two electrodes.

In some implementations, the second subset of electrodes is selected based on the position of the second subset of electrodes relative to the first subset of electrodes. In some implementations, the first subset of electrodes is selected based on the position of the first subset of electrodes relative to the neural target. In some implementations, the method further comprises selecting a second subset of electrodes to maximise a distance between any one electrode of the first subset of electrodes and any one electrode of the second subset of electrodes.

In some implementations, the method further comprises selecting, from the plurality of electrodes, a third subset of electrodes, based on the measured response of the neural target, and providing stimulation energy to the third subset of electrodes to stimulate the neural target. The third subset of electrodes may comprise at least one electrode that is not a member of the first subset of electrodes.

According to another aspect of the present technology, there is provided a paddle electrode assembly for an implantable medical device. The paddle electrode assembly is configured to be electrically coupled to an implantable stimulation device comprising stimulation circuitry and measurement circuitry. The paddle electrode assembly comprises a lead body having a proximal end and a distal end, and a paddle disposed at the distal end of the lead body. The paddle comprises a paddle body, comprising a ventral surface and a dorsal surface, and a plurality of electrodes including a first group of one or more electrodes arranged on the ventral surface of the paddle body, and a second group of one or more electrodes arranged on the dorsal surface of the paddle body. The lead body is configured to electrically couple each electrode of the plurality of electrodes to the stimulation circuitry of the implantable stimulation device, and the lead body is configured to electrically couple each electrode of the plurality of electrodes to the measurement circuitry of the implantable stimulation device.

In some implementations, the lead body is configured to electrically couple each electrode of the second group of electrodes to the measurement circuitry of the implantable stimulation device. In some implementations, the second group of electrodes includes at least two electrodes.

In some implementations, the plurality of electrodes are positioned on the ventral side of the paddle body. In some implementations, the paddle electrode assembly further comprises one or more electrodes positioned on the dorsal side of the paddle body. In some implementations, the paddle body has an elongated form defined by a rostro-caudal length and a medial-lateral width.

In some implementations, the plurality of electrodes comprises a plurality of rostro-caudally aligned rows of electrodes. In some implementations, the plurality of electrodes comprises a first rostro-caudally aligned row of electrodes and a second rostro-caudally aligned row of electrodes, and the second row of electrodes is offset from the first row of electrodes on the paddle body in a medial-lateral direction. In some implementations, the electrodes of the first rostro-caudally aligned row of electrodes are offset from the electrodes of the second rostro-caudally aligned row of electrodes in the rostro-caudal direction.

In some implementations, the plurality of rostro-caudally aligned rows of electrodes comprises a first electrode row, positioned at a left lateral side of the paddle body, a second electrode row, positioned at a medial midline of the paddle body, and a third electrode row, positioned at a right lateral side of the paddle body. In some implementations, the first electrode row comprises seven electrodes, the second electrode row comprises eight electrodes and the third electrode row comprise seven electrodes.

In some implementations, at least one electrode of the plurality of electrodes has an electrode surface area that is polygonal. In some implementations, at least one electrode of the plurality of electrodes has an electrode surface area that is hexagonal.

In some implementations, the paddle further comprises at least one electrode having an electrode surface that is substantially shorter in the rostro-caudal direction than in the medial-lateral direction. In some implementations, the at least one electrode is configured to be a recording electrode. In some implementations, the at least one electrode is a wire electrode.

In some implementations, the paddle further comprises a shield ring electrode pair. The shield ring electrode pair comprises a centre electrode, and a ring electrode which at least partially encircles the centre electrode.

References herein to estimation, determination, comparison and the like are to be understood as referring to an automated process carried out on data by a processor operating to execute a predefined procedure suitable to effect the described estimation, determination and/or comparison step(s). The technology disclosed herein may be implemented in hardware (e.g., using digital signal processors, application specific integrated circuits (ASICs) or field programmable gate arrays (FPGAs)), or in software (e.g., using instructions tangibly stored on non-transitory computer-readable media for causing a data processing system to perform the steps described herein), or in a combination of hardware and software. The disclosed technology can also be embodied as computer-readable code on a computer-readable medium. The computer-readable medium can include any data storage device that can store data which can thereafter be read by a computer system. Examples of the computer-readable medium include read-only memory (“ROM”), random-access memory (“RAM”), magnetic tape, optical data storage devices, flash storage devices, or any other suitable storage devices. The computer-readable medium can also be distributed over network-coupled computer systems so that the computer-readable code is stored and/or executed in a distributed fashion.

### BRIEF DESCRIPTION OF THE DRAWINGS

One or more implementations of the invention will now be described with reference to the accompanying drawings, in which:

FIG. 1 schematically illustrates an implanted spinal cord stimulator in a patient, according to one implementation of the present technology;

FIG. 2 is a block diagram of the stimulator of FIG. 1, according to one implementation of the present technology;

FIG. 3 is a schematic illustrating interaction of an implanted stimulator with a nerve in a patient, according to one implementation of the present technology;

FIG. 4 illustrates the typical form of an ECAP of a healthy subject, as recorded at a single measurement electrode referenced to the system ground, according to one implementation of the present technology;

FIG. 5 illustrates an idealised activation plot for one posture of a patient, according to one implementation of the present technology;

FIG. 6 illustrates the variation in the activation plots with changing posture of a patient, according to one implementation of the present technology;

FIG. 7 is a schematic illustrating elements and inputs of a closed loop neurostimulation system, according to one implementation of the present technology;

FIG. 8 is a block diagram of a neuromodulation system, according to one implementation of the present technology;

FIG. 9 is a block diagram illustrating the data flow of a neuromodulation therapy system such as the system of FIG. 8, according to one implementation of the present technology;

FIG. 10 illustrates the stimulation fields for an electrode of a paddle electrode assembly and an electrode of an epidural electrode array, according to one implementation of the present technology;

FIG. 11 illustrates current flow paths of two stimulus electrodes and one passive electrode, according to one implementation of the present technology;

FIG. 12 illustrates six electrodes of a paddle electrode assembly, according to one implementation of the present technology;

FIG. 13 illustrates the wavelength of an ECAP with regard to the separation distance of two measurement electrodes, according to one implementation of the present technology;

FIGS. 14A to 14D illustrate rows of six electrodes configured in different arrangements of stimulating and measurement electrodes, according to one or more implementations of the present technology;

FIG. 15A schematically illustrates the ventral surface of a paddle, according to one implementation of the present technology;

FIG. 15B schematically illustrates the dorsal surface of the paddle of FIG. 15A, according to one implementation of the present technology;

FIG. 16 is a flowchart illustrating a method for selecting electrode configurations for stimulating a neural target, according to an implementation of the present technology;

FIGS. 17A, 17B and 17C illustrate portions of three separate paddles, wherein each paddle has the same surface area and same number of electrodes, according to one or more implementations of the present technology;

FIG. 18 illustrates an array of rectangular electrodes of a paddle electrode assembly, according to one implementation of the present technology;

FIG. 19 illustrates an array of rectangular electrodes of a paddle electrode assembly, according to another implementation of the present technology;

FIG. 20 illustrates a shield ring electrode, according to one implementation of the present technology;

FIG. 21 illustrates electrodes of a paddle electrode assembly that includes shield ring electrodes, according to one implementation of the present technology;

FIG. 22 illustrates electrodes of a paddle electrode assembly that includes shield ring electrodes, according to another implementation of the present technology;

FIG. 23 illustrates electrodes of a paddle electrode assembly that includes shield ring electrodes, according to yet another implementation of the present technology;

FIG. 24 illustrates electrodes of a paddle electrode assembly that includes shield ring electrodes, according to yet another implementation of the present technology;

FIG. 25 illustrates electrodes of a paddle electrode assembly that includes shield ring electrodes, according to yet another implementation of the present technology;

FIG. 26A schematically illustrates the ventral surface of a paddle, according to one implementation of the present technology; and

FIG. 26B schematically illustrates the dorsal surface of the paddle of FIG. 26A, according to one implementation of the present technology.

### DETAILED DESCRIPTION OF THE PRESENT TECHNOLOGY

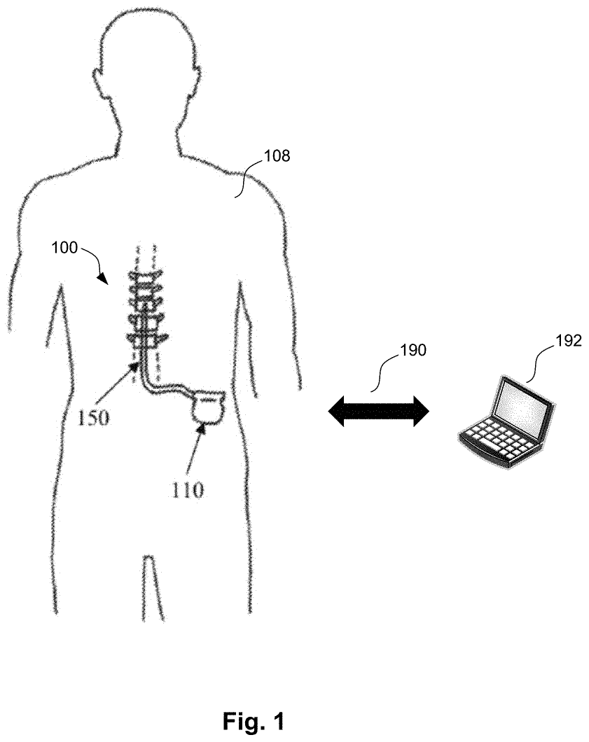

### FIG. 1—Implanted Stimulator in Patient

FIG. 1 schematically illustrates an implanted spinal cord stimulator 100 in a patient 108, according to one implementation of the present technology. Stimulator 100 comprises an electronics module 110 implanted at a suitable location. In one implementation, stimulator 100 is implanted in the patient's lower abdominal area or posterior superior gluteal region. In other implementations, the electronics module 110 is implanted in other locations, such as a flank or sub-clavicular. Stimulator 100 further comprises an electrode array 150 implanted within the epidural space and connected to the module 110 by a suitable lead. The electrode array 150 may comprise one or more electrodes such as electrode pads on a paddle electrode assembly, circular (e.g., ring) electrodes surrounding the body of the lead (as known as an electrode array of an epidural electrode assembly), conformable electrodes, cuff electrodes, segmented electrodes, or any other type of electrodes capable of forming unipolar, bipolar or multipolar electrode configurations for stimulation and measurement. The electrodes may pierce or affix directly to the tissue itself.

Numerous aspects of the operation of implanted stimulator 100 may be programmable by an external computing device 192, which may be operable by a user such as a clinician or the patient 108. Moreover, implanted stimulator 100 serves a data gathering role, with gathered data being communicated to external device 192 via a transcutaneous communications channel 190. Communications channel 190 may be active on a substantially continuous basis, at periodic intervals, at non-periodic intervals, or upon request from the external device 192. External device 192 may thus provide a clinical interface configured to program the implanted stimulator 100 and recover data stored on the implanted stimulator 100. This configuration is achieved by program instructions collectively referred to as the Clinical Programming Application (CPA) and stored in an instruction memory of the clinical interface.

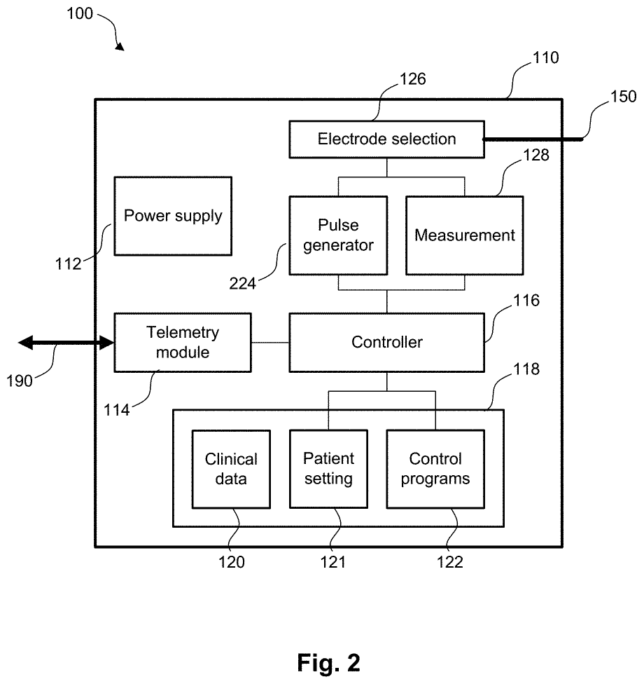

### FIG. 2—Block Diagram of Stimulator

FIG. 2 is a block diagram of the stimulator 100, according to one implementation of the present technology. Electronics module 110 contains a battery 112 and a telemetry module 114. In implementations of the present technology, any suitable type of transcutaneous communication 190, such as infrared (IR), radiofrequency (RF), capacitive and inductive transfer, may be used by telemetry module 114 to transfer power and/or data to and from the electronics module 110 via communications channel 190. Module controller 116 has an associated memory 118 storing one or more of clinical data 120, patient settings 121, control programs 122, and the like. Controller 116 controls a pulse generator 124 to generate stimuli, such as in the form of pulses, in accordance with the patient settings 121 and control programs 122. Electrode selection module 126 switches the generated pulses to the selected electrode(s) of electrode array 150, for delivery of the pulses to the tissue surrounding the selected electrode(s). Measurement circuitry 128, which may comprise an amplifier 129 and/or an analog-to-digital converter (ADC), is configured to process measurements of neural responses sensed by electrode(s) of the electrode array 150 as selected by electrode selection module 126.

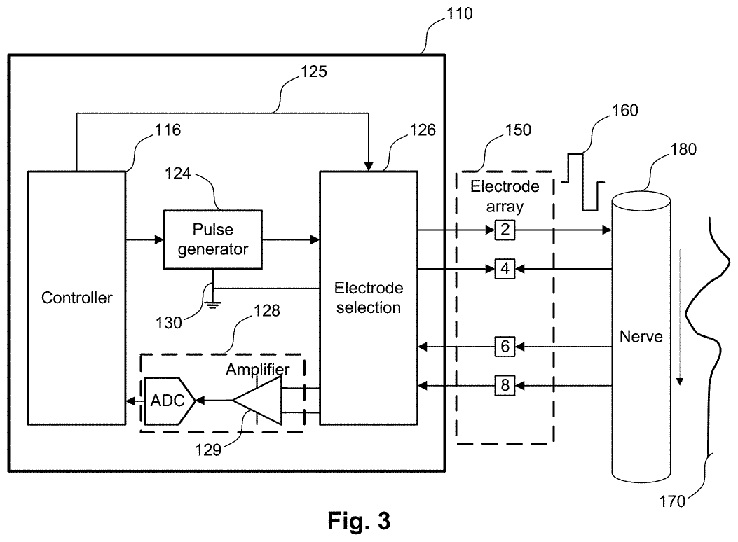

### FIG. 3—Interaction of Implanted Stimulator

FIG. 3 is a schematic illustrating interaction of the implanted stimulator 100 with a nerve 180 in the patient 108, according to one implementation of the present technology. In the implementation illustrated in FIG. 3 the nerve 180 may be located in the spinal cord, however in alternative implementations the stimulator 100 may be positioned adjacent any desired neural tissue including a peripheral nerve, visceral nerve, parasympathetic nerve or a brain structure.

Electrode selection module 126 is configured by the controller 116 via control signal 125. In response to configuration by the controller 116, electrode selection module 126 selects a stimulus electrode 2 of electrode array 150 through which to deliver a pulse from the pulse generator 124. A pulse may comprise one or more phases, e.g. a biphasic stimulus pulse 160 comprises two phases. The electrode selection module 126 selects a stimulus electrode (cathode) 2 to deliver the pulse to surrounding tissue including nerve 180. Electrode selection module 126 also selects a return electrode (anode) 4 of the electrode array 150 for stimulus charge recovery in each phase, to maintain a zero net charge transfer. The use of two electrodes in this manner for delivering and recovering current in each stimulus phase is referred to as bipolar stimulation. Alternative embodiments may apply other forms of bipolar stimulation, or may use a greater number of stimulus electrodes. Electrode selection module 126 is illustrated as connecting to a ground 130 of the pulse generator 124 to enable stimulus charge recovery via the return electrode 4. However, other connections for charge recovery may be used in other implementations.

Delivery of an appropriate stimulus from stimulus electrodes 2 and 4 to the nerve 180 evokes a neural response comprising an evoked compound action potential 170 (ECAP) which will propagate along the nerve 180 as illustrated, for therapeutic purposes, which in the case of a spinal cord stimulator for chronic pain may be to create paraesthesia at a desired location. To this end, the stimulus electrodes 2 and 4 are used to deliver stimuli periodically at any therapeutically suitable frequency, for example 30 Hz, although other frequencies may be used including frequencies as high as the kHz range. In alternative implementations, stimuli may be delivered in a non-periodic manner such as in bursts, or sporadically, as appropriate for the patient 108. To “fit” the stimulator 100 to the patient 108, a clinician may cause the stimulator 100 to deliver stimuli of various configurations which seek to produce a sensation that is experienced by the user as paraesthesia. When a stimulus configuration is found which evokes paraesthesia in a location and of a size which is congruent with the area of the patient's body affected by pain, the clinician nominates that configuration for ongoing use.

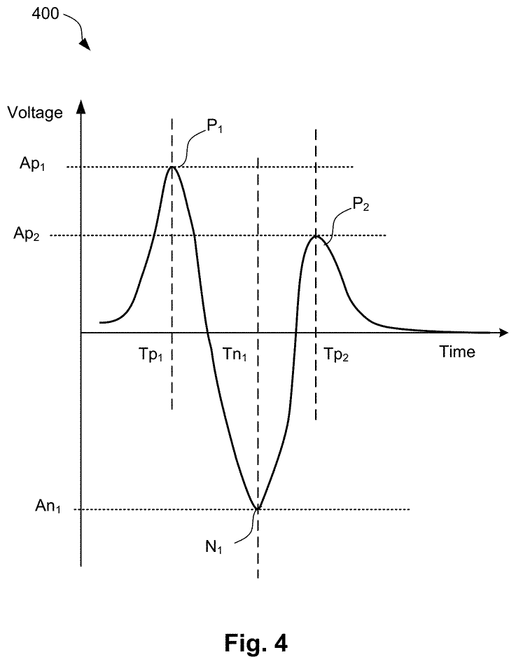

### FIG. 4—Typical Form of an ECAP

FIG. 4 illustrates the typical form of an ECAP of a healthy subject, as sensed by a single electrode referenced to the system ground 130, according to one implementation of the present technology. The shape and duration of the single-ended ECAP 400 shown in FIG. 4 is predictable because it is a result of the ion currents produced by the ensemble of fibres depolarising and generating action potentials (APs) in response to stimulation. The evoked action potentials (EAPs) generated synchronously among a large number of fibres sum to form the ECAP 400. The propagation velocity of the AP on each fibre is determined largely by the diameter of that fibre. The ECAP 400 generated from the synchronous depolarisation of a group of similar fibres comprises a positive peak P1, then a negative peak N1, followed by a second positive peak P2. This shape is caused by the region of activation passing the measurement electrode as the action potentials propagate along the individual fibres.

The ECAP may be recorded differentially using two measurement electrodes, as illustrated in FIG. 3. Differential ECAP measurements are less subject to common-mode noise on the surrounding tissue than single-ended ECAP measurements. Depending on the polarity of recording, a differential ECAP may take an inverse form to that shown in FIG. 4, i.e. a form having two negative peaks N1 and N2, and one positive peak P1. Alternatively, depending on the distance between the two measurement electrodes, a differential ECAP may resemble the time derivative of the form 400, or more generally the difference between the form 400 and a time-delayed copy thereof.

The ECAP 400 may be parametrised by any suitable parameter(s) of which some are indicated in FIG. 4. The amplitude of the positive peak P1 is Ap₁ and occurs at time Tp₁. The amplitude of the positive peak P2 is Ap₂ and occurs at time Tp₂. The amplitude of the negative peak P1 is An₁ and occurs at time Tn₁. The peak-to-peak amplitude is Ap₁+An₁. A recorded ECAP will typically have a maximum peak-to-peak amplitude in the range of microvolts and a duration of 2 to 3 ms.

The stimulator 100 is further configured to sense the existence and intensity of ECAPs 170 propagating along nerve 180, whether such ECAPs are evoked by the stimulus from electrodes 2 and 4, or otherwise evoked. To this end, any electrodes of the array 150 may be selected by the electrode selection module 126 to serve as measurement recording electrode 6 and measurement reference electrode 8, whereby the electrode selection module 126 selectively connects the chosen electrodes to the inputs of the measurement circuitry 128. Thus, signals sensed by the measurement electrodes 6 and 8 are passed to the measurement circuitry 128, which may comprise an amplifier and an analog-to-digital converter (ADC). The measurement circuitry 128 for example may operate in accordance with the teachings of International Patent Application Publication No. WO2012155183 by the present applicant, the contents of which are incorporated herein by reference.

Neural responses obtained from the measurement electrodes 6, 8 via measurement circuitry 128 are processed by controller 116 to obtain information regarding the effect of the applied stimulus upon the nerve 180. In some implementations, neural responses are processed by controller 116 in a manner which extracts and stores one or more parameters from each response or group of responses. In one such implementation, the parameter comprises a peak-to-peak ECAP amplitude in microvolts (μV). For example, the neural responses may be processed to determine the peak-to-peak ECAP amplitude in accordance with the teachings of International Patent Publication No. WO 2015/074121, the contents of which are incorporated herein by reference. Alternative implementations may extract and store an alternative parameter from the response to be stored, or may extract and store two or more parameters from the response.

Stimulator 100 applies stimuli over a potentially long period such as days, weeks, or months and during this time may store parameters of neural responses, stimulation settings, paraesthesia target level, and other operational parameters in memory 118. To effect suitable SCS therapy, stimulator 100 may deliver tens, hundreds or even thousands of stimuli per second, for many hours each day. Each neural response or group of responses generates one or more parameters such as a measure of the amplitude of the neural response. Stimulator 100 thus may produce such data at a rate of tens or hundreds of Hz, or even kHz, and over the course of hours or days this process results in large amounts of clinical data which may be stored in the clinical data store 120 of memory 118. Memory 118 is however necessarily of limited capacity and care is thus required to select compact data forms for storage into the memory 118, to ensure that the memory 118 is not exhausted before such time that the data is expected to be retrieved wirelessly by external device 192, which may occur only once or twice a day, or less.

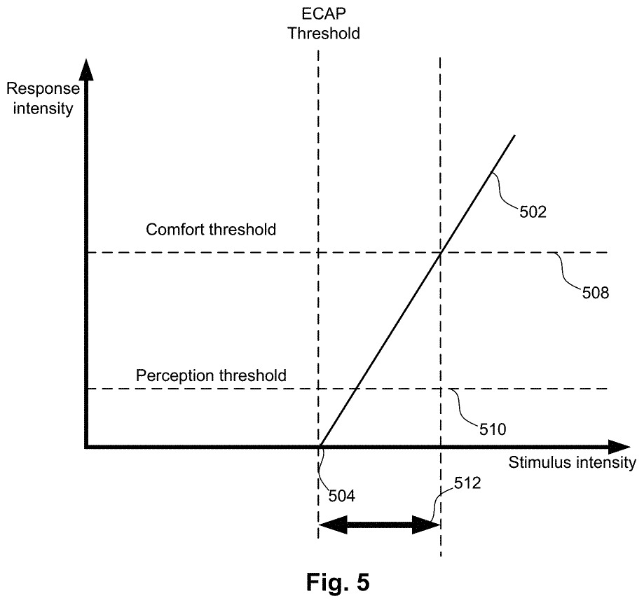

### FIG. 5—Activation Plot

An activation plot, or growth curve, is an approximation to the relationship between stimulus intensity (e.g. an amplitude of the current pulse 160) and intensity of neural response 170 resulting from the stimulus (e.g. an ECAP amplitude). FIG. 5 illustrates an idealised activation plot 502 for one posture of the patient 108, according to one implementation of the present technology. The activation plot 502 shows a linearly increasing ECAP amplitude for stimulus amplitude values above a threshold 504, referred to as the ECAP threshold. The ECAP threshold 504 exists because of the binary nature of fibre recruitment; if the field strength is too low, no fibres will be recruited. However, once the field strength exceeds a threshold, fibres begin to be recruited, and their individual evoked action potentials are independent of the strength of the field. The ECAP threshold 504 therefore reflects the field strength at which significant numbers of fibres begin to be recruited, and the increase in response intensity with stimulus amplitude above the ECAP threshold reflects increasing numbers of fibres being recruited. Below the ECAP threshold 504, the ECAP amplitude may be taken to be zero. Above the ECAP threshold 504, the activation plot 502 has a positive, approximately constant slope indicating a linear relationship between stimulus amplitude and the ECAP amplitude. Such a relationship may be modelled as:

$$y = \{ \begin{matrix}S ( s - T ) , & s \geq T \\ 0 , & s < T\end{matrix} \qquad ( 1 )$$

where s is the stimulus amplitude, y is the ECAP amplitude, T is the ECAP threshold and S is the slope of the activation plot (referred to herein as the patient sensitivity). The slope S and the ECAP threshold T are the key parameters of the activation plot 502.

FIG. 5 also illustrates a comfort threshold 508, which is an ECAP amplitude above which the patient 108 experiences uncomfortable or painful stimulation. FIG. 5 also illustrates a perception threshold 510. The perception threshold 510 corresponds to an ECAP amplitude that is perceivable by the patient. There are a number of factors which can influence the position of the perception threshold 510, including the posture of the patient. Perception threshold 510 may correspond to a stimulus amplitude that is greater than the ECAP threshold 504, as illustrated in FIG. 5, if patient 108 does not perceive low levels of neural activation. Conversely, the perception threshold 510 may correspond to a stimulus amplitude that is less than the ECAP threshold 504, if the patient has a high perception sensitivity to lower levels of neural activation than can be detected in an ECAP, or if the signal to noise ratio of the ECAP is low.

For effective and comfortable operation of an implantable neuromodulation device such as the stimulator 100, it is desirable to maintain stimulus amplitude within a therapeutic range. A stimulus amplitude within a therapeutic range is above the ECAP threshold 504 and evokes an ECAP amplitude that is below the comfort threshold 508. In principle, it would be straightforward to measure these limits and ensure that stimulus amplitude, which may be closely controlled, always falls within the therapeutic range 512. However, the activation plot, and therefore the therapeutic range 512, varies with the posture of the patient 108.

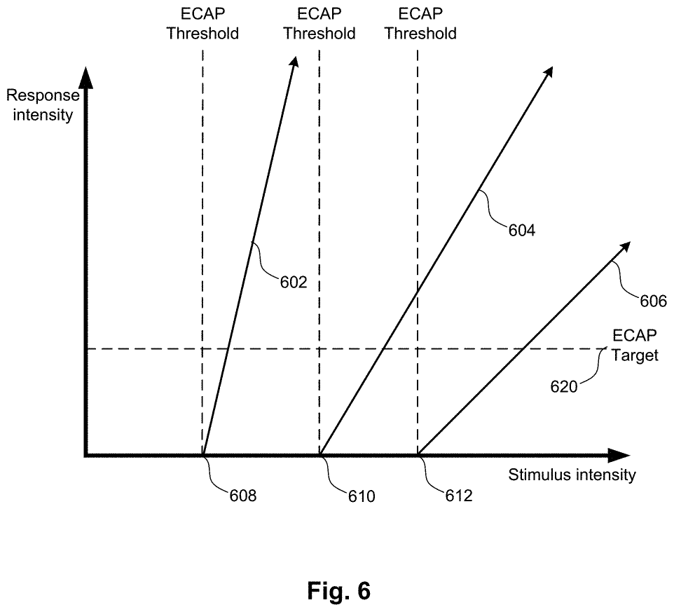

FIG. 6—Variation in Activation Plots with Changing Posture

FIG. 6 illustrates the variation in the activation plots with changing posture of the patient, according to one implementation of the present technology. A change in posture of the patient may cause a change in impedance of the electrode-tissue interface or a change in the distance between electrodes and the neurons. While the activation plots for only three postures, 602, 604 and 606, are shown in FIG. 6, the activation plot for any given posture can lie between or outside the activation plots shown, on a continuously varying basis depending on posture. Consequently, as the patient's posture changes, the ECAP threshold changes, as indicated by the ECAP thresholds 608, 610 and 612 for the respective activation plots 602, 604, and 606. Additionally, as the patient's posture changes, the slope of the activation plot also changes, as indicated by the varying slopes of activation plots 602, 604 and 606. In general, as the distance between the stimulus electrodes and the spinal cord increases, the ECAP threshold increases and the slope of the activation plot decreases. The activation plots 602, 604, and 606 therefore correspond to increasing distance between stimulus electrodes and spinal cord, and decreasing patient sensitivity.

To keep the applied stimulus intensity within the therapeutic range as patient posture varies, in some implementations an implantable neuromodulation device such as the stimulator 100 may adjust the applied stimulus amplitude based on a feedback variable that is determined from one or more extracted ECAP parameters. In one implementation, the device may adjust the stimulus amplitude to maintain the extracted ECAP amplitude at a target response intensity. For example, the device may calculate an error between a target ECAP value and a measured ECAP amplitude, and adjust the applied stimulus intensity to reduce the error as much as possible, such as by adding the scaled error to the current stimulus intensity. A neuromodulation device that operates by adjusting the applied stimulus intensity based on an extracted ECAP parameter is said to be operating in closed loop mode and will also be referred to as a closed loop neural stimulus (CLNS) device. By adjusting the applied stimulus intensity to maintain the extracted ECAP amplitude at an appropriate target response intensity, such as an ECAP target 620 illustrated in FIG. 6, a CLNS device will generally keep the stimulus intensity within the therapeutic range as patient posture varies.

A CLNS device comprises a stimulator that takes a stimulus intensity value and converts it into a neural stimulus comprising a sequence of electrical pulses according to a predefined stimulation pattern. The stimulation pattern is characterised by multiple parameters including stimulus intensity (amplitude), pulse width, number of phases, order of phases, number of stimulus electrode poles (two for bipolar, three for tripolar etc.), and stimulus rate or frequency. At least one of the stimulus parameters, usually the stimulus intensity, is controlled by the feedback loop.

In an example CLNS system, a user (e.g. the patient or a clinician) sets a target neural response value, and the CLNS performs proportional-integral-differential (PID) control. In some implementations, the differential contribution is disregarded and the CLNS system uses a first order integrating feedback loop. The stimulator produces stimulus in accordance with a stimulus intensity parameter, which evokes a neural response in the patient. The evoked neural response (e.g. an ECAP) is detected and its amplitude measured by the CLNS and compared to the target neural response value.

The measured neural response amplitude, and its deviation from the target neural response value, is used by the feedback loop to determine possible adjustments to the stimulus intensity parameter to maintain the neural response at the target value. If the target value is properly chosen, the patient receives consistently comfortable and therapeutic stimulation through posture changes and other perturbations to the stimulus/response behaviour.

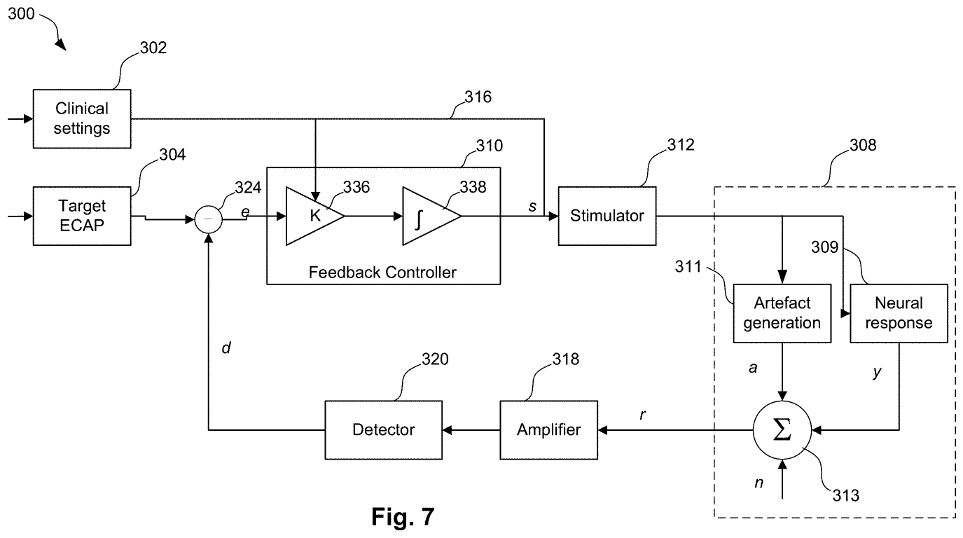

### FIG. 7—CLNS System

FIG. 7 is a schematic illustrating elements and inputs of a closed loop neurostimulation system 300, according to one implementation of the present technology. The system 300 comprises a stimulator 312 which converts a stimulus intensity parameter (for example a stimulus current value) s, in accordance with a set of predefined stimulus parameters, to a neural stimulus comprising a sequence of electrical pulses on the stimulus electrodes (not shown in FIG. 5). According to one implementation, the predefined stimulus parameters comprise the number and order of phases, the number of stimulus electrode poles, the pulse width, and the stimulus rate or frequency.

The generated stimulus crosses from the electrodes to the spinal cord, which is represented in FIG. 7 by the dashed box 308. The box 309 represents the evocation of a neural response y by the stimulus as described above. The box 311 represents the evocation of an artefact signal a, which is dependent on stimulus intensity and other stimulus parameters, as well as the electrical environment of the measurement electrode. Various sources of noise n may add to the evoked response y at the summing element 313 before the evoked response is measured, including electrical noise from external sources such as 50 Hz mains power; electrical disturbances produced by the body such as neural responses evoked not by the device but by other causes such as peripheral sensory input, EGG, EMG; and electrical noise from amplifier 318.

The neural recruitment arising from the stimulus is affected by mechanical changes, including posture changes, walking, breathing, heart beat and so on. Mechanical changes may cause impedance changes, or changes in the location and orientation of the nerve fibres relative to the electrode array(s). As described above, the intensity of the evoked response provides a measure of the recruitment of the fibres being stimulated. In general, the more intense the stimulus, the more recruitment and the more intense the evoked response. An evoked response typically has a maximum amplitude in the range of microvolts, whereas the applied stimulus to evoke the response is typically several volts.

The total response signal r (including evoked neural response, artefact, and noise) is amplified by the signal amplifier 318 and then measured by the detector 320. The detector 320 outputs a measured response intensity d. In one implementation, the neural response intensity comprises an ECAP value. The measured response intensity d is then compared to a target ECAP value (set by the target ECAP controller 304) by the comparator 324 to produce an error value e. The error value e is input into the feedback controller 310.

The comparator 324 compares the ECAP value of the total response signal r to the target ECAP value as set by the target ECAP controller 304 and provides an indication of the difference between the ECAP value of the total response signal r and the target ECAP value to the feedback controller 310. This difference is the error value, e.

The feedback controller 310 calculates an adjusted stimulus intensity parameter, s, with the aim of maintaining a measured response intensity d equal to the target ECAP value. Accordingly, the feedback controller 310 adjusts the stimulus intensity parameters to minimise the error value, e. In one implementation, the controller 310 utilises a first order integrating function, using a gain element 336 and an integrator 338, in order to provide suitable adjustment to the stimulus intensity parameter s. According to such an implementation, an adjustment δs to the current stimulus intensity parameter s may be computed by the feedback controller 310 as

s=∫Kedt (2)

A target ECAP value is input to the comparator 324 via the target ECAP controller 304. In one embodiment, the target ECAP controller 304 provides an indication of a specific target ECAP value. In another embodiment, the target ECAP controller 304 provides an indication to increase or to decrease the present target ECAP value. The target ECAP controller 304 may comprise an input into the neural stimulus device, via which the patient or clinician can input a target ECAP value, or indication thereof. The target ECAP controller 304 may comprise memory in which the target ECAP value is stored, and provided to the comparator 324.

A clinical settings controller 302 provides clinical parameters to the system, including the gain K for the gain controller 336 and the stimulation parameters for the stimulator 312. The clinical settings controller 302 may be configured to adjust the gain value, K, of the gain controller 336 to adapt the feedback loop to patient sensitivity. The clinical settings controller 302 may comprise an input into the neural stimulus device, via which the patient or clinician can adjust the clinical settings. The clinical settings controller 302 may comprise memory in which the clinical settings are stored, and are provided to components of the system 300.

In some implementations, two clocks (not shown) are used, being a stimulus clock operating at the stimulus frequency (e.g. 60 Hz) and a sample clock for sampling the measured response r (for example, operating at 10 kHz). As the detector 320 is linear, only the stimulus clock affects the dynamics of the CLNS 300. On the next stimulus clock cycle, the stimulator 312 outputs a stimulus in accordance with the adjusted stimulus intensity s. Accordingly, there is a delay of one stimulus clock cycle before the stimulus is updated in light of the error value e.

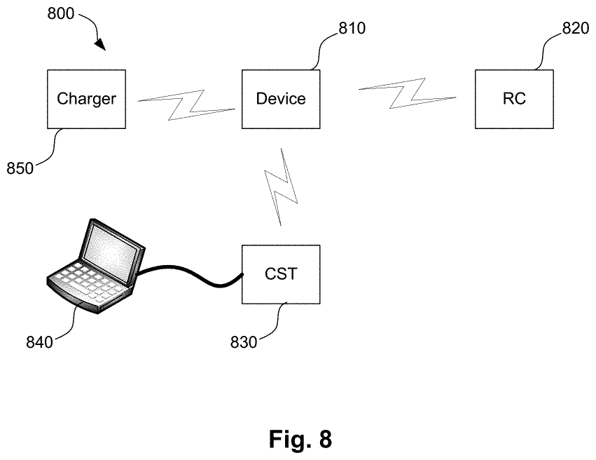

### FIG. 8—Neuromodulation System

FIG. 8 is a block diagram of a neuromodulation system 800, according to one implementation of the present technology. The neuromodulation system 800 is centred on a neuromodulation device 810. In one example, the neuromodulation device 810 may be implemented as the stimulator 100 of FIG. 1, implanted within a patient (not shown). The neuromodulation device 810 is connected wirelessly to a remote controller (RC) 820. The remote controller 820 is a portable computing device that provides the patient with control of their stimulation in the home environment by allowing control of the functionality of the neuromodulation device 810, including one or more of the following functions: enabling or disabling stimulation; adjustment of stimulation intensity; and selection of a stimulation control program from the control programs stored on the neuromodulation device 810.

The charger 850 is configured to recharge a rechargeable power source of the neuromodulation device 810. The recharging is illustrated as wireless in FIG. 8 but may be wired in alternative implementations.

The neuromodulation device 810 is wirelessly connected to a Clinical System Transceiver (CST) 830. The wireless connection may be implemented as the transcutaneous communications channel 190 of FIG. 1. The CST 830 acts as an intermediary between the neuromodulation device 810 and the Clinical Interface (CI) 840, to which the CST 830 is connected. A wired connection is shown in FIG. 8, but in other implementations, the connection between the CST 830 and the CI 840 is wireless.

The clinical interface 840 may be implemented as the external computing device 192 of FIG. 1. The CI 840 is configured to program the neuromodulation device 810 and recover data stored on the neuromodulation device 810. This configuration is achieved by program instructions collectively referred to as the Clinical Programming Application (CPA) and stored in an instruction memory of the clinical interface 840.

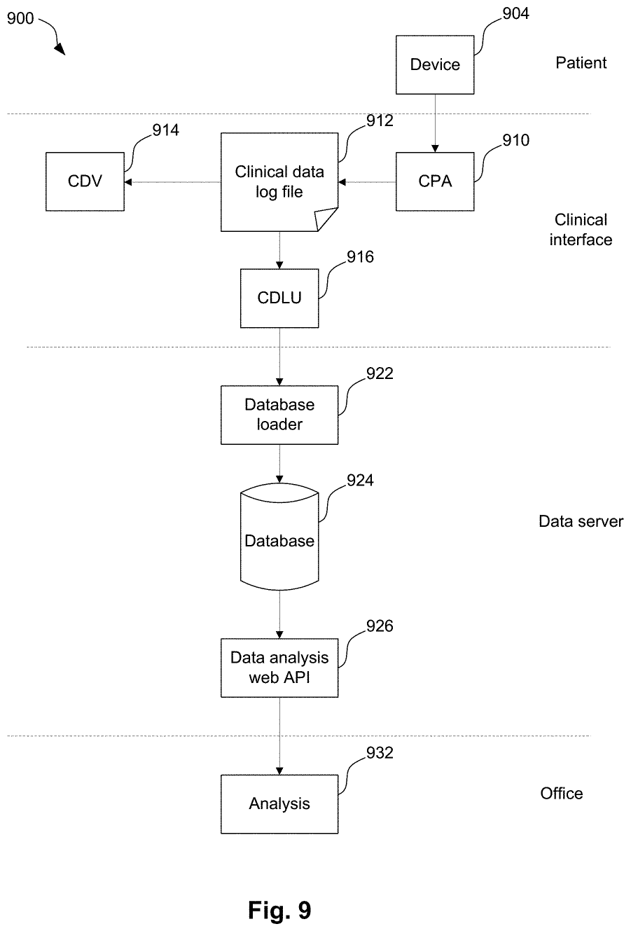

### FIG. 9—Data Flow of Neuromodulation Therapy System

FIG. 9 is a block diagram illustrating the data flow 900 of a neuromodulation therapy system such as the system 800 of FIG. 8, according to one implementation of the present technology. Neuromodulation device 904, once implanted within a patient, applies stimuli over a potentially long period such as weeks or months and records neural responses, stimulation settings, paraesthesia target level, and other operational parameters, discussed further below. Neuromodulation device 904 may comprise a Closed Loop Stimulator (CLS), in that the recorded neural responses are used in a feedback arrangement to control stimulation settings on a continuous or ongoing basis. To effect suitable SCS therapy, neuromodulation device 904 may deliver tens, hundreds or even thousands of stimuli per second, for many hours each day. The feedback loop may operate for most or all of this time, by obtaining neural response recordings following every stimulus, or at least obtaining such recordings regularly. Each recording generates a feedback variable such as a measure of the amplitude of the evoked neural response, which in turn results in the feedback loop changing the stimulation parameters for a following stimulus. Neuromodulation device 904 thus produces such data at a rate of tens or hundreds of Hz, or even kHz, and over the course of hours or days this process results in large amounts of clinical data. This is unlike past neuromodulation devices such as open-loop SCS devices which lack any ability to record any neural response.

When brought in range with a receiver, neuromodulation device 904 transmits data, e.g. via telemetry module 114, to a clinical programming application (CPA) 910 installed on a clinical interface. In one implementation, the clinical interface is the CI 840 of FIG. 8. The data can be grouped into two main sources: (1) Data collected in real-time during a programming session; (2) Data downloaded from a stimulator after a period of non-clinical use by a patient. CPA 910 collects and compiles the data into a clinical data log file 912.

All clinical data transmitted by the neuromodulation device 904 may be compressed by use of a suitable data compression technique before transmission by telemetry module 114 and/or before storage into the Clinical Data storage 120 to enable storage by neuromodulation device 904 of higher resolution data. This higher resolution allows neuromodulation device 904 to provide more data for post-analysis and more detailed data mining for events during use. Alternatively, compression enables faster transmission of standard-resolution clinical data.

The clinical data log file 912 is manipulated, analysed, and efficiently presented by a clinical data viewer (CDV) 914 for field diagnosis by a clinician, field clinical engineer (FCE) or the like. CDV 914 is a software application installed on the Clinical Interface (CI). In one implementation, CDV 914 opens one Clinical Data Log file 912 at a time. CDV 914 is intended to be used in the field to diagnose patient issues and optimise therapy for the patient. CDV 914 may be configured to provide the user or clinician with a summary of neuromodulation device usage, therapy output, and errors, in a simple single-view page immediately after log files are compiled upon device connection.

Clinical Data Uploader 916 is an application that runs in the background on the CI, that uploads files generated by the CPA 910, such as the clinical data log file 912, to a data server. Database Loader 922 is a service which runs on the data server and monitors the patient data folder for new files. When Clinical Data Log files are uploaded by Clinical Data Uploader 916, database loader 922 extracts the data from the file and loads the extracted data to Database 924.

The data server further contains a data analysis web API 926 which provides data for third-party analysis such as by the analysis module 932, located remotely from the data server. The ability to obtain, store, download and analyse large amounts of neuromodulation data means that the present technology can: improve patient outcomes in difficult conditions; enable faster, more cost effective and more accurate troubleshooting and patient status; and enable the gathering of statistics across patient populations for later analysis, with a view to diagnosing aetiologies and predicting patient outcomes.

### Tripolar Stimulation

The controller 116 configures the electrode selection module 126 to select a plurality of the electrodes of the electrode array to provide stimulation to the nerve 180. The plurality of electrodes selected to provide stimulation represents a stimulus electrode configuration (SEC).

Various stimulus electrode configurations (SECs) may be configured depending upon the type and location of the stimulation desired by the controller 116. For tripolar stimulation, each SEC is tripolar, comprising a stimulus electrode that acts primarily as a cathode, sinking stimulus current, with the two neighbouring return electrodes on either side of the stimulus electrode acting primarily as anodes, sourcing return currents. Tripolar stimulus electrode configurations are described in more detail in International Patent Publication No. WO2020082118 by the present applicant, the contents of which are incorporated herein by reference.

Other embodiments may utilise four stimulus electrodes to deliver quadrupolar stimulation. For example offset quadrupolar stimulation may be delivered whereby the suprathreshold stimulus component is delivered by one electrode and returned by three electrodes.

Other embodiments may utilise five or more stimulus electrodes to deliver pentapolar stimulation or to deliver stimuli from a greater number of electrode poles. In some embodiments, the electrode selection module 126 may be configured by the controller 116 to adaptively select the number of stimulus electrodes in order to seek which of a tripolar, quadrupolar, pentapolar etc configuration best minimises second cathode stimulation, by switching between such stimulation modes.

The current borne by the first stimulus electrode may in some embodiments be divided equally between the two or more other stimulus electrodes. For example in the case of tripolar stimulation having two return electrodes each return electrode may carry 50% of the current delivered by the first stimulus electrode. Alternatively, the current borne by the first stimulus electrode may be divided unequally between the two or more other stimulus electrodes while maintaining each at a sub-threshold level, and for example such unequal currents may be configured to take an inequality which minimises stimulus artefact in accordance with the teachings of the present Applicant's International Patent Application Publication No. WO 2017/219096, the contents of which are incorporated herein by reference.

The current carried by each respective stimulus electrode may be controlled by providing a respective current source for each electrode configured to drive the desired current through that respective electrode in each phase of the stimulus. Alternatively, a respective current source may be provided between adjacent pairs of stimulus electrodes to effect differential drive of the desired current through each electrode. Such embodiments may be particularly applicable when unequal current sharing between the stimulus electrodes is required. Alternatively, one or more of the stimulus electrodes may be grounded to serve as a passive return electrode for example.

The first stimulus electrode in some embodiments is interposed between the two or more other electrodes, for example upon an epidural lead array. In alternative embodiments the first stimulus electrode may be positioned to one side of both or all of the return electrodes. Such embodiments recognise that it is desirable to record an ECAP as close as possible to the site of stimulation, before ECAP propagation effects of a compound response cause dispersion and amplitude reduction of the initial neural response. In such embodiments, the return electrode stimulus components may be maintained at an equal level or at least at a respective sub-threshold level by driving each return electrode with a respective current source.

### Measurement Electrodes

The electrode selection module 126 may further comprise circuitry to select one or more electrodes of the electrode array 150 to be measurement electrodes and measurement circuitry 128, configured to obtain one or more recordings of a neural response, such as an ECAP, evoked by the stimulus provided by the stimulus electrodes.

In one embodiment, two electrodes, a recording electrode and its reference electrode, are used by the measurement circuitry 128 to measure an ECAP. Preferably, the reference electrode is placed in a location that would result in the highest possible signal-to-artefact ratio value.

Embodiments of the electronics module 110 may utilise any suitable ECAP detector to assess the neural recordings to assess recruitment, such as an ECAP vector detector. The vector detector may for example utilise a four-lobed or five-lobed matched filter template in accordance with the teachings of the present applicant's International Patent Publication No. WO2015/074121, the contents of which are incorporated herein by reference. Particularly preferred embodiments may apply a stimulus in accordance with the present invention, detect an evoked response by using an ECAP vector detector, and use the output of the detector to control a stimulation feedback loop.

### Electrode Array Type

The implanted electronics module 110 may be coupled to an electrode array 150 implanted within the epidural space of the patient and connected to the module 110 by a suitable lead. The electrode array may comprise an epidural electrode array in which ring-shaped electrodes are arranged along an elongated, substantially cylindrical lead. In some implementations, both the stimulating and recording electrodes may be arranged along the lead of an epidural electrode assembly.

Alternatively, the electrode assembly may comprise a paddle electrode assembly. A paddle electrode assembly, otherwise called a ‘paddle’, may comprise a flat surface area, substantially rectangular in shape, upon which flat electrodes are arranged. The paddle may comprise an array of electrodes arranged in one or more rows such that, when implanted in the patient, the one or more rows of electrodes face the neural target of the patient and are aligned along the rostro-caudal line of the patient.

In some embodiments, the electrode assembly comprises a combination of an epidural electrode assembly and a paddle electrode assembly. For example, the electrode assembly may comprise electrodes arranged on a paddle surface, and electrodes arranged around an epidural lead that is coupled to the paddle surface.

An epidural electrode assembly and a paddle electrode assembly typically differ in terms of their respective implantation mechanism, the span of the electrodes and the electrode geometry.

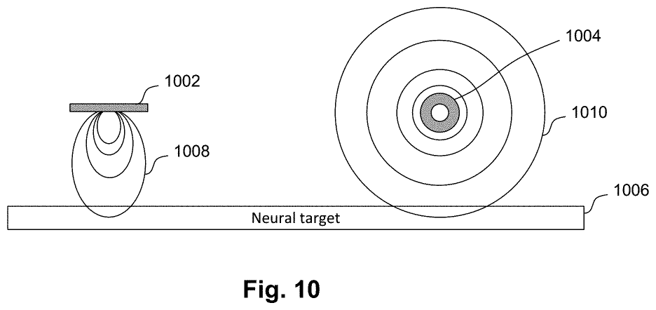

### FIG. 10—Stimulation Fields of Paddle Electrodes and Epidural Electrodes

FIG. 10 illustrates the stimulation fields for an electrode 1002 of a paddle electrode assembly and an electrode 1004 of an epidural electrode assembly, according to one implementation of the present technology. Electrode 1002 is coupled to the surface of a paddle, and has a substantially flat surface from which the stimulation field projects. In one example, electrode 1002 is a circular disc shaped electrode. In contrast, electrode 1004 is substantially cylindrical in shape and is coupled to a lead which extends through the centre of electrode 1004.

The geometric differences between a paddle electrode and an epidural lead electrode result in differences between the stimulation fields of the two electrodes. The stimulation field 1008 of paddle electrode 1002 is unidirectional, projecting substantially perpendicular to the flat surface of the electrode 1002. This means that the paddle electrode 1002 may be positioned to direct the main portion of the stimulation energy of the stimulation field 1008 towards a neural target 1006, such as the dorsal column of a patient.

The stimulation field 1010 of epidural electrode 1004 is circumferential stimulation, projecting outwards from the outer perimeter of the electrode 1004. Some of the stimulation field 1010 of electrode 1004 is received by the neural target 1006; however, a significant portion of the stimulation field 1010 is not received by the neural target 1006. The stimulation energy that is not received by the neural target 1006 may be wasted, or may interfere with the desired stimulation of the neural target 1006.

In some implementations, the unidirectional stimulation field of the paddle electrode 1002 is preferred as it enables the efficient transfer of stimulation energy from the electrode 1002 to the neural target 1006, whilst reducing the undesirable stimulation of non-targeted regions.

In some embodiments, the use of a paddle electrode, rather than an epidural lead electrode, may require half the stimulation current to achieve the same level of stimulation of a neural target. For example, where a charge injection of 2 μC is required through an epidural lead, a lower injection of 1 μC may be expected to be required through a paddle lead.

### Measurement Electrodes Separate to the Paddle

In one implementation an electronics module of a stimulation device may be coupled to a paddle electrode assembly, comprising one or more electrodes configured to provide stimulation. The electronics module may be further coupled to a measurement lead, which comprises one or more electrodes coupled to measurement circuitry by the electrode selection module 126. The measurement lead may be coupled to the paddle electrode assembly, for example, coupled to the distal end of the paddle. Alternatively, the measurement lead may be separate from the paddle.

### Electrode Selection Module

In some implementations it is advantageous to be able to configure one or more electrodes on a paddle electrode assembly to act as either a measurement electrode or a stimulus electrode. In some implementations, it is advantageous for an electrode to act as a measurement electrode for one or more stimulation cycles, then the same electrode acts as a stimulus electrode for one or more further stimulation cycles.

In one implementation, each electrode of the paddle electrode assembly may be coupled, by the electrode selection module 126, to stimulation circuitry (e.g. the pulse generator 124), measurement circuitry 128 or neither the stimulation circuitry or measurement circuitry. The electrode selection module 126 may comprise one or more multiplexors to couple electrodes of the paddle to either the stimulation circuitry or the measurement circuitry.

For ease of reference, one may refer to an electrode that is coupled to stimulation circuitry as a stimulus electrode. Similarly, one may refer to an electrode that is coupled to measurement circuitry as a measurement electrode. An electrode that is coupled to neither the stimulation circuitry nor the measurement circuitry may be referred to as an unused or passive electrode. In some embodiments, the electrode selection module 126 may couple an electrode to stimulation circuitry for one or more stimulation cycles. The electrode selection module 126 may then couple the electrode to measurement circuitry for one or more subsequent stimulation cycles. Accordingly, an electrode may function as a stimulus electrode for a period of time, then the same electrode may function as a measurement electrode. Similarly, an electrode may function as a measurement electrode for a period of time, then the same electrode may function as a stimulus electrode.

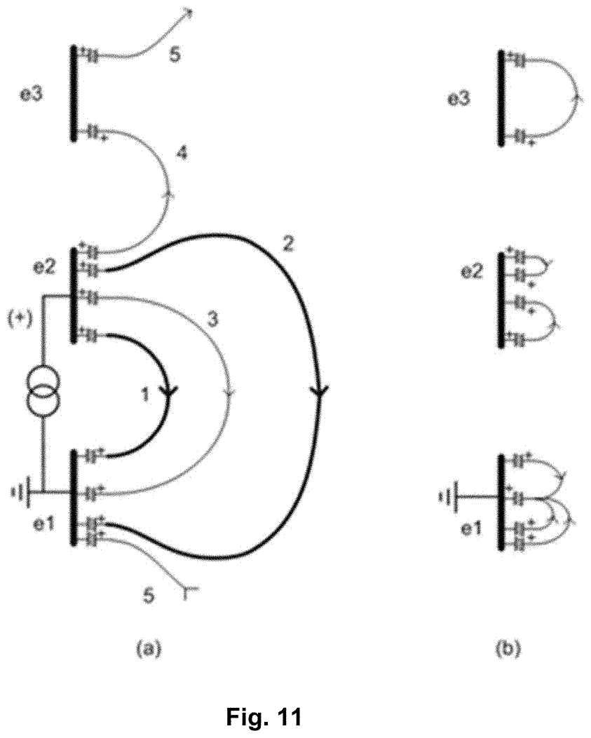

### FIG. 11—Split Electrode Artefacts

During stimulation, electric fields are produced by the stimulus electrodes. These electric fields result in field gradients that extend across the surfaces of the electrodes, and may surround the stimulus electrodes. These field gradients can cause charge accumulation in the surrounding electrodes, and this charge accumulation may dissipate for a period that extends well into the recording period. This charge dissipation may appear as an artefact in the measured ECAP value, and adversely affect the accuracy of the ECAP measurement.

FIG. 11 illustrates current flow paths of two stimulus electrodes and one passive electrode, according to one implementation of the present technology. More particularly, FIG. 11 diagram (a) illustrates the current flow path in the simplified case of two stimulus electrodes, e1 and e2, and one passive electrode, e3, during a stimulation pulse. FIG. 11 diagram (b) illustrates the same example after the stimulation pulse.

Electrodes e1 and e2 are configured as stimulus electrodes, with current passing from electrode e2 to electrode e1, as illustrated by lines 1, 2 and 3. Some charge flows out the other end of electrode e2 to electrode e3, as illustrated by line 4.

The current flow labelled 5 may be 100 times smaller than current flows 1, 2 and 3, but it leaves passive electrode e3 with a significant displaced charge. FIG. 11 diagram (a) and (b) illustrate how a completely passive metal structure becomes charged during stimulation of nearby electrodes. Accordingly, a passive metal structure may come to host a displaced population of mobile species in the Gouy-Stern-Chapman layer.

In an embodiment in which electrode e3 is configured as a measurement electrode to measure the ECAP response evoked in response to the stimulation of electrodes e1 and e2, the stored charge in electrode e3 dissipates during the ECAP measurement, resulting in a distortion of the measured ECAP value. This distortion is called the split electrode artefact. Split electrode artefact results from a charge flowing into one end of a contact and out the other leading to charge stored that then dissipates during ECAP measurement.

In some embodiments, the magnitude of the split electrode artefact is dependent on the geometry of the electrode assembly and dependent upon the arrangement of electrodes on the electrode assembly. Accordingly, it may be desirable to determine an electrode assembly which mitigates the adverse impact of split electrode artefacts arising from dissipating charge accumulation.

### Passive Electrode Between Stimulating and Measurement Electrode

To ameliorate the effect of a split electrode artefact, the electrodes on the electrode assembly may be configured to increase the separation between stimulus electrodes and measurement electrodes. In one example, the electrodes on the electrode assembly may be configured such that there is an unused (passive) electrode situated between the stimulus electrodes and measurement electrodes.

This method of ameliorating the split electrode artefact is described in more detail in International Patent Publication No. WO2020082126 by the present applicant, the contents of which are incorporated herein by reference.

In some embodiments, the configuration of an unused electrode between the stimulus electrodes and the measurement electrodes on an electrode assembly, can restrict which electrodes are available on the electrode assembly to be used as measurement electrodes. Accordingly, this may require the configuration of the electrodes such that the distance between the measurement electrodes is reduced.

### Signal to Artefact Ratio (SAR)

It is preferable to have a high signal to artefact ratio with regard to the measured response of the tissue, e.g. the measured ECAP response. In some embodiments, it is preferable for the artefact component of the measured ECAP response to be less than 10 μV in response to a stimulation of 1 μC (200 us, 5 mA) with attenuating factor ω=1 (as defined below).

The signal to artefact ratio (SAR) may be described by equation (3).

$$S A R = \frac{S}{A} \qquad ( 3 )$$

where S describes the ECAP component of the signal recorded by an electrode pair,
A is the measured artefact in the configuration.

The size of the recorded ECAP varies with the separation between electrodes as the ECAP voltage along the cord has an approximate sinusoidal shape. Moving them closer, or further away from this value reduces the magnitude of the recorded ECAP signal.

A theoretical value for the expected signal value for a recording pair can be derived using the reference ECAP value. This value captures the effect of planar/cylindrical electrodes on signal value. Using the lowest clinically useful values ensures the results are relevant in situations where low ECAP signal is expected. This value does not capture how the signal is attenuated by the spatial arrangement of the recording electrode pair.

A simple model that captures this is described by equation (4).

S=ωR (4)
where S described the ECAP component of the signal recorded by an electrode pair, R is the reference ECAP value and co is an attenuating factor described by equation (5).

$$ω = sin ( \frac{π d}{18 mm} ) \qquad ( 5 )$$

where d describes the distance between the two recording electrodes.

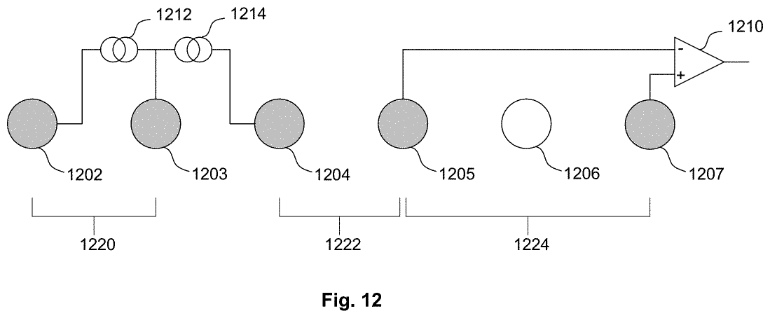

### FIG. 12—Preferred Electrode Separation

FIG. 12 illustrates six electrodes, 1202 to 1207, of a paddle electrode assembly, according to one implementation of the present technology. The electrodes are configured to be implanted in the patient such that the row of six electrodes 1202 to 1207 are substantially in alignment along the rostro-caudal axis of the patient. Electrodes 1202, 1203 and 1204 are coupled to stimulation circuitry 224, via the electrode selection module 126, and are therefore configured to function as stimulation electrodes. The stimulus electrodes, 1202, 1203 and 1204, receive stimulation energy from the stimulation circuitry 224 via current sources 1212 and 1214.

Electrodes 1205 and 1207 are coupled to measurement circuitry 128 via the electrode selection module 126, and are therefore configured to function as recording electrodes. Recording electrodes 1205 and 1207 are configured to enable measurement of an ECAP response of the neural tissue. The ECAP response of the tissue is measured by determining a voltage across electrodes 1205 and 1207, as amplified by amplifier 1210.

Distance 1220 indicates the stimulus electrode separation, being the separation between stimulus electrodes. Distance 1222 indicates the stimulus to measurement electrode separation, being the separation between the stimulus electrode that is nearest either of the measurement electrodes, and the nearest measurement electrode. The signal to artefact ratio (SAR) improves as the recording site moves further away from the triad of stimulus electrodes. As the distance between the stimulus and recording electrodes increased, in both the rostro-caudal and medial-lateral directions, the SAR improved.

Distance 1224 indicates the measurement electrode separation, being the separation between the measurement electrodes 1205 and 1207.

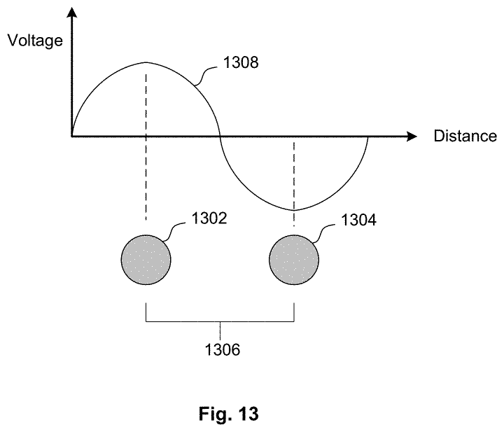

### FIG. 13—Preferred Separation of Measurement Electrodes

FIG. 13 illustrates the wavelength of an ECAP with regard to the separation distance of two measurement electrodes, according to one implementation of the present technology. The measurement electrodes comprise a recording electrode 1302 and a reference electrode 1304.

In the example of FIG. 13, the fundamental frequency of the ECAP 1308 is approximately 1 kHz, with a propagation velocity of approximately 40 m/s, with a 40 mm wavelength. A preferred separation distance 1306 of a recording electrode from a reference electrode is at half this wavelength, at 15 to 22 millimetres. A preferred separation between a recording electrode and a reference electrode may be 14 to 21 millimetres, which may correspond to a distance of 2 to 3 electrodes on a paddle electrode assembly. More particularly, a preferred separation between a recording electrode and a reference electrode may be 18 millimetres.

### Lateral Adjustment of Stimulation

A neural target, such as a neural cord of a spine, may extend along a rostro-caudal axis of the patient, and extend along a medial-lateral axis of the patient (i.e. left-hand side to right-hand side). In some implementations, there is a preferred site on the neural target for stimulation in order to achieve the desired therapeutic benefit for the patient. Preferred site of stimulation may change with movement of the patient, or movement of the paddle relative to the patient. To allow for shifting of the preferred stimulation site relative to the paddle, either during fitting or in use, it may be desirable to provide stimulation from a site on the paddle that is not along the midline, extending along the rostro-caudal axis, of the paddle assembly. This enables a lateral adjustment of the site of stimulation along the medial-lateral axis of the paddle assembly. This lateral adjustment provides the ability to treat pain on one side of the neural target and to compensate when anatomical midline of the patient is not at perceptual midline of the patient.

A paddle assembly that comprises a plurality of electrodes at various positions along the rostro-caudal axis, as well as a plurality of electrodes at various positions along the medial-lateral axis provides allows for rostro-caudal adjustment of the stimulation site as well as lateral adjustment of the stimulation site.

In one implementation, a paddle assembly comprises multiple rows of electrodes including, a centre row of electrodes extending along the rostro-caudal direction, and a row of electrodes to the left of the centre row, and a row of electrodes to the right of the centre row.

### Lateral Measurement of ECAP

In implementations in which the neural target extends in a rostro-caudal direction, it is preferable that the triad of stimulation electrodes is also aligned in a rostro-caudal direction to align with the fibres of the neural target. In some implementations, it is desirable that the pair of measurement electrodes are aligned rostro-caudally to align with DC fibres. In some implementations, it is desirable for a paddle electrode to provide the ability to measure the ECAP from a position that is lateral (in the medial-lateral axis) of the stimulation site.

Accordingly, for some implementations, it may be preferable for the electrode assembly to comprise an array of electrodes, which provide for various stimulation site options along the rostro-caudal direction, as well as along the medial-lateral direction.

### Dimensions of a Paddle Electrode Assembly

A paddle electrode assembly comprises an array of electrodes arranged on a substantially flat, two-dimensional surface. As used herein, the term ‘arranged on’ is intended to encompass, but not be limited to, a variety of manufacturing techniques including being ‘affixed to’, ‘attached to’, ‘mounted on’, ‘fastened to’, ‘embedded into’, ‘impressed in’, ‘made integral to’, or ‘formed integrally with’.

The geometry of a paddle electrode assembly is defined with reference to the intended configuration of the paddle electrode assembly when it is implanted in a subject to provide stimulation to a neural target. The rostro-caudal dimension of the paddle extends along the length of the paddle and is in alignment with the direction of extension of the neural target (e.g. along the spine of the subject). The ventral surface of the paddle is the flat surface directed towards the neural target, whereas the dorsal surface of the paddle faces in the opposite direction to the ventral surface and away from the neural target. The paddle is also defined by a lateral axis which defines the width of the paddle. The paddle may be referred to as having a midline, a left-hand side and a right-hand side.

### Rows of Electrodes

It is desirable to consider the relationship between the arrangement of stimulation and recording electrodes on a paddle and the split electrode artefact that may arise during use. A consideration of this relationship may inform the development of a preferred arrangement of stimulating and recording electrodes on a paddle suitable for use in closed-loop spinal cord stimulation (SCS).

As noted above, in some implementations it is preferable to utilise tripolar stimulation, using three stimulation electrodes. Additionally, it may be preferable to use three stimulation electrodes that are proximate to each other, in accordance with the preferred stimulation electrode spacing. Furthermore, using three stimulation electrodes that are substantially aligned along a rostro-caudal direction of the neural target, when implanted, provides a single stimulating site for the neural target.

Considering a paddle electrode assembly that comprises a row of five electrodes substantially aligned along a rostro-caudal direction, if the paddle electrode is configured to use the middle set of three electrodes for tripolar stimulation, then the two remaining electrodes, which are located at the distal and proximal ends of the paddle, can be used as measurement electrodes. However, these measurement electrodes will be separated by a distance that is greater than the preferred separation of measurement electrodes for recording the ECAP response. Accordingly, for some implementations it is preferable for a paddle to comprise more than five electrodes aligned in a row along a rostro-caudal direction.

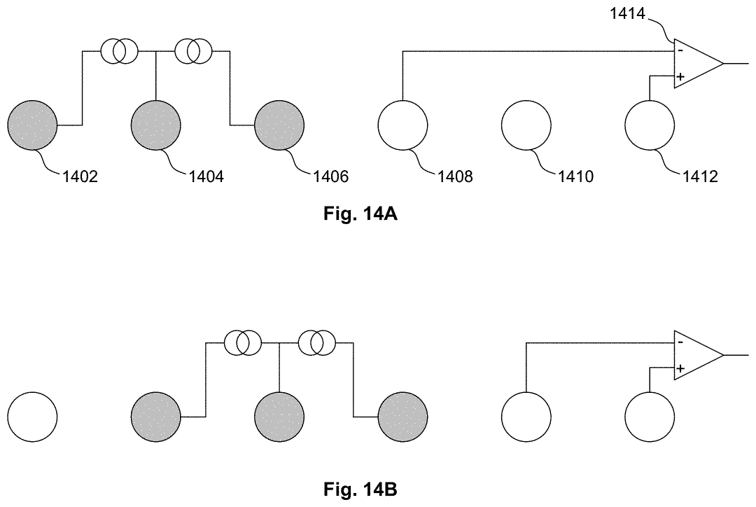

### FIGS. 14A to 14D—Row of Six Electrodes

FIGS. 14A to 14D illustrate rows of 6 electrodes configured in different arrangements of stimulating and measurement electrodes, according to one or more implementations of the present technology. Electrodes that have been connected, via an electrode selection module of the electronics module, to stimulation circuitry are stimulus electrodes. Stimulating electrodes are illustrated as shaded circles. Electrodes that have been connected to the measurement circuitry are measurement electrodes, and are illustrated as unshaded circles.

FIG. 14A illustrates three stimulus electrodes 1402, 1404 and 1406, and two measurement electrodes, 1408 and 1412, electrically coupled to amplifier 1414. In the configuration illustrated in FIG. 14A, electrode 1410 is unused. Measurement electrodes 1408 and 1412 are separated by the preferred measurement electrode separation of 2 to 3 electrodes.

In each of the electrode configurations illustrated in FIGS. 14A to 14D, three adjacent electrodes are configured to provide tripolar stimulation, and there are two electrodes on at least one side of the three adjacent stimulus electrodes which may be configured to be measurement electrodes. Accordingly, the configuration of a row of six electrodes, aligned along the rostro-caudal direction, which may be configured to be either stimulus electrodes or measurement electrodes, provides more stimulation site options than a row of five electrodes.

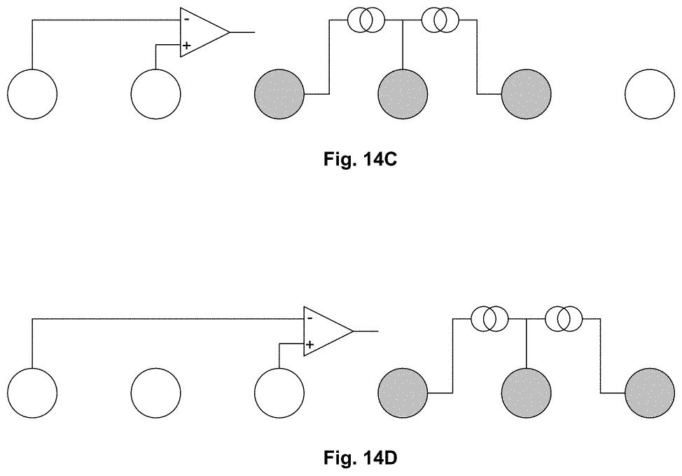

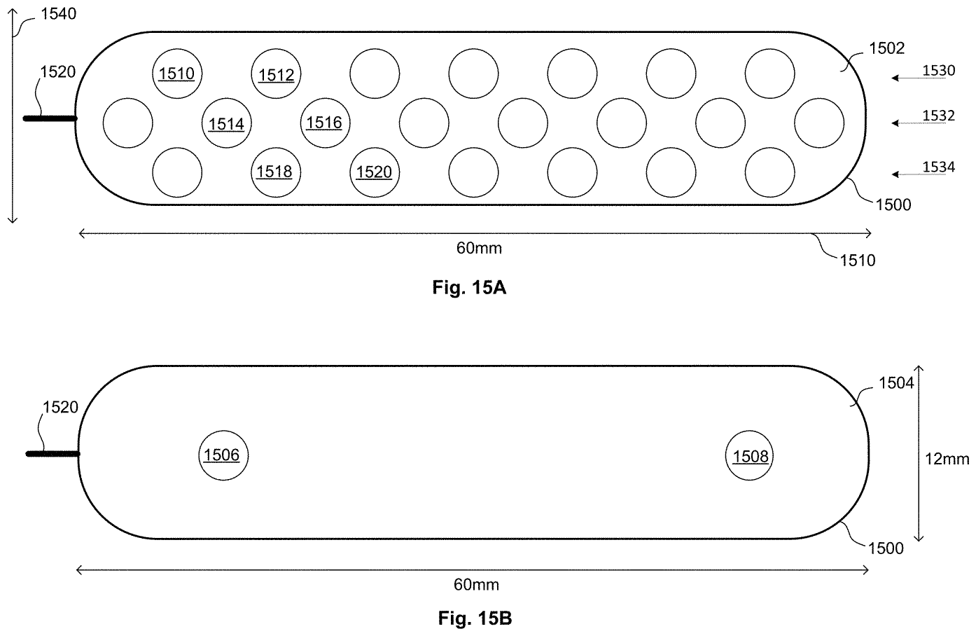

### FIGS. 15A and 15B—Example Paddle Electrode Assembly

FIG. 15A schematically illustrates the ventral surface of a paddle 1500, according to one implementation of the present technology. The paddle 1500 comprises a paddle body 1502 and a plurality of electrodes attached or affixed to the paddle body 1502. The paddle 1500 is integrated with a paddle lead 1520 which is configured to electrically couple each of the plurality of electrodes of the paddle 1500 to an electronics module, such as electronics module 110.

The ventral surface of the paddle body 1502 comprises a first group of electrodes, comprising 22 electrodes arranged in three rows along the rostro-caudal direction of the paddle body. The middle row of electrodes, indicated by arrow 1532, comprises eight electrodes. The left-hand row of electrodes, indicated by arrow 1530, and the right-hand row of electrodes, indicated by arrow 1534, comprise 7 electrodes each.

In this implementation illustrated in FIG. 15, each of the 22 electrodes on the ventral surface of the paddle 1500 may be configured to be stimulus electrodes or measurement electrodes. As noted previously, an electrode selection module, such as electrode selection module 126, electrically couples electrodes of the paddle to either stimulating circuitry or measurement circuitry. Alternatively, one or more electrodes of the paddle may be not connected to either the stimulating or measurement circuitry, and may therefore be passive, or unused.

### Dorsal Side Electrodes

FIG. 15B schematically illustrates the dorsal surface of paddle 1500, according to one implementation of the present technology. The dorsal surface 1504 of the paddle 1500 comprises a second group of electrodes. In the implementation illustrated in FIG. 15B, the second group of electrodes comprises dorsal-side electrodes 1506 and 1508. In other implementations, the second group of electrodes may comprise one or more dorsal-side electrodes.

Electrodes 1506 and 1508 may each be configured to be stimulus electrodes or measurement electrodes, or may be configured to be passive (unused) electrodes. In one embodiment, the electronics module 126 configures one or both of dorsal-side electrodes 1506 and 1508 to act as a measurement electrode.

Advantageously, a dorsal-side electrode that is configured to be a measurement electrode is positioned substantially out of the field of stimulation generated by one or more stimulation electrodes on the ventral side of the paddle. Such a measurement electrode may function as an indifferent electrode, unaffected either by stimulation or any neural response. An indifferent electrode, when used as a reference electrode, turns a differential ECAP measurement between two measurement electrodes into effectively a single-ended ECAP measurement, as would be observed if the reference electrode were at the system ground 130, while still largely rejecting common-mode noise. Configuring a dorsal-side electrode as a reference electrode also allows greater freedom to configure a ventral-side electrode as the recording electrode so as ameliorate the effects of a split-electrode artefact on the recording electrode.

### 7-8-7 Electrode Array

The number of electrical connections from the electronics module to the paddle lead may define the number of electrodes that the paddle may include. In an implementation in which the electronics module supports 24 electrical contacts, a preferred configuration of electrodes on a paddle electrode assembly comprises a midline row of eight electrodes, a left-hand side row of seven electrodes, a right-hand side row of seven electrodes, and two electrodes positioned on the dorsal side of the paddle. This may be referred to as a 7-8-7 electrode array.

### Electrode Configurations

The electrode selection module 126 configures each of the electrodes of the paddle electrode assembly to act as a stimulus electrode, a measurement electrode or a passive (unused) electrode. The electrodes that the electrode selection module 126 configures to act as measurement electrodes are collectively called a measurement electrode configuration (MEC).

A measurement electrode configuration (MEC) comprises two electrodes for differential ECAP recording. The measurement electrode connected to the positive terminal of the measurement amplifier 129, via the electrode selection module 126, is referred to as the recording electrode, while the measurement electrode connected to the negative terminal of the measurement amplifier 129, via the electrode selection module 126, is referred to as the reference electrode.

The electrodes that the electrode section module 126 configures to act as stimulus electrodes are collectively called a stimulus electrode configuration (SEC). A SEC comprises three electrodes: a cathode electrode, configured to sink stimulus current; and two anode electrodes, configured to source return currents. The cathode electrode may be located in a position between the two anode electrodes. The three stimulus electrodes may all be located in the same row of electrodes in the rostro-caudal direction.

The electrode selection module 126, acting under controller of the controller 116 of the electronics module 110 selects the MEC and SEC to achieve the desired closed loop neural stimulation of the neural target. Furthermore, in response to the measured ECAP, movement of the neural target, or movement of the paddle in relation to the neural target, or a change in the desired stimulation intensity, the electrode selection module 126, acting under controller of the controller 116 of the electronics module 110 adjusts the MEC and SEC to achieve the desired stimulation intensity at the desired stimulation site.

In one embodiment, the controller 116 configures the electrode selection module 126 to select the SEC to provide stimulation to a preferred site on the neural target, then the controller 116 configures the electrode selection module 126 to select the MEC in order to obtain the most accurate ECAP measurement.

In one embodiment, the electrode selection module 126 selects the MEC such that the reference electrode and the recording electrode are in the same row along the rostro-caudal direction (e.g. the same caudal column). In one embodiment, the electrode selection module 126 selects the MEC such that there is a preferred spacing between the measurement electrodes. In one embodiment, the preferred measurement electrode spacing is 18 millimetres. In one embodiment, the electrode selection module 126 selects the MEC such that the positive recording electrode sits between the stimulating triad and the negative recording electrode.

### Staggered Electrodes

In some implementations, it is preferable to stagger the position of the electrodes, in the rostro-caudal direction, on the paddle. In the implementation illustrated in FIG. 15A, line 1510 indicates the length of the paddle along the rostro-caudal direction, and line 1540 indicates the width of the paddle along the medial-lateral direction.

The electrodes positioned on the ventral surface 1502 of the paddle 1500 are positioned in three rows which are each substantially aligned with the rostro-caudal length of the paddle 1500, indicated by line 1510. The electrodes in each row are positioned in a staggered position relative to the electrodes of the adjacent row or rows. For example, electrode 1514 is positioned substantially equidistant between electrode 1510 and 1512 in the rostro-caudal direction. Similarly, electrode 1518 is positioned equidistant between electrodes 1514 and 1516 in the rostro-caudal direction.

A staggered arrangement of electrodes provides more stimulation site options along the rostro-caudal dimension of the paddle assembly. For example, in a situation in which it is desirable to provide stimulation at a site on the neural target between electrode 1510 and electrode 1512, it may be effective to provide stimulation by electrode 1514 which is positioned half-way between electrode 1510 and electrode 1512.

In addition to providing more stimulation site options in the rostro-caudal direction, a staggered arrangement of electrodes aids in increasing the separation between the electrodes without increasing the width of the paddle.

### Aligned Electrodes

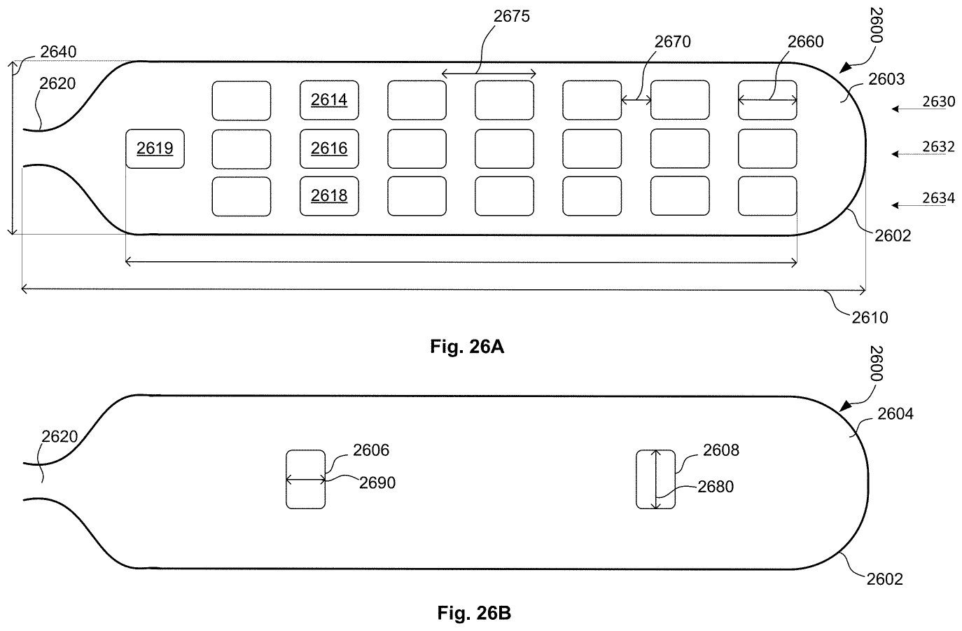

In some implementations, it is preferable to align the position of the electrodes, in the rostro-caudal direction, on the paddle body. FIG. 26A schematically illustrates the ventral surface 2603 of a paddle 2600, according to one implementation of the present technology. FIG. 26B schematically illustrates the dorsal surface 2604 of paddle 2600, according to one implementation of the present technology.

The paddle 2600 comprises a paddle body 2602 and a plurality of electrodes attached to, affixed to, or integrated with, the paddle body 2602. The paddle 2600 is integrated with a paddle lead 2620 which is configured to electrically couple each of the plurality of electrodes of the paddle 2600 to an electronics module, such as electronics module 110. Line 2610 indicates the rostro-caudal dimension of the paddle 2600, and line 2640 indicates the medial-lateral dimension of the paddle 2600.

The ventral surface 2603 of the paddle body 2602 comprises 22 electrodes arranged in three rows along the rostro-caudal direction. The middle row, indicated by arrow 2632, comprises eight electrodes. The left-hand row, indicated by arrow 2630, and the right-hand row, indicated by arrow 2634, each comprise 7 electrodes.

In the implementation illustrated in FIG. 26A, each of the 22 electrodes on the ventral surface 2603 of the paddle body 2602 may be configured to be stimulus electrodes or measurement electrodes. As noted previously, an electrode selection module, such as electrode selection module 126, electrically couples electrodes of the paddle 2600 to either stimulating circuitry or measurement circuitry. Alternatively, one or more electrodes of the paddle 2600 may be not connected to either the stimulating or measurement circuitry, and may therefore be passive, or unused.

In the implementation illustrated in FIG. 26A, the three row-wise arrangements of the electrodes positioned on the ventral surface 2603 of the paddle 2600, as indicated by arrows 2630, 2632, and 2634, are mutually substantially aligned along the rostro-caudal length of the paddle 2600. That is, the electrodes in each row are positioned in an aligned position, along the rostro-caudal dimension, relative to the electrodes of the adjacent row or rows. For example, electrodes 2614, 2616 and 2618 are positioned substantially in alignment with regard to the rostro-caudal dimension of the paddle 2600, such that these electrodes are positioned at substantially the same distance from the paddle lead 2620. Electrode 2619 is not aligned, in the rostro-caudal dimension, with any other electrode on the paddle body 2602.

A rostro-caudally aligned arrangement of electrodes on the ventral surface 2603 of the paddle body 2602, as shown in FIG. 26A, is advantageous, for example by providing a spatially compact arrangement of electrodes on the paddle body 2602.

The dorsal surface 2604 of the paddle body 2602 comprises two dorsal side electrodes 2606 and 2608. In other embodiments, the dorsal surface 2604 of the paddle body 2602 may comprise one or more dorsal side electrodes. Each electrode on the dorsal surface 2604 of the paddle body 2602 may be configured to be a stimulus electrode, a measurement electrode, or a passive (unused) electrode. Accordingly, the paddle body 2602 may be configured to have one or more dorsal-side electrodes acting as measurement electrodes and/or one or more dorsal-side electrodes acting as stimulating electrodes.

In one embodiment, the electrode selection module 126, electrically couples electrodes of the paddle to either stimulating circuitry or measurement circuitry. Alternatively, one or more electrodes of the paddle 2600 may be not connected to either the stimulating or measurement circuitry, and may therefore be passive, or unused.

In a preferred embodiment, the ventral side electrodes have a substantially rectangular shaped surface area with which to provide stimulation, or to measure an ECAP. In a preferred embodiment, the dorsal side electrodes have a substantially rectangular shaped surface area with which to provide stimulation, or to measure an ECAP.

In a preferred embodiment, the length of the paddle along the rostro-caudal dimension, as indicated by line 2610, is 62 millimeters with an error margin of 0.5 millimeters. In a preferred embodiment, the width of the paddle along the medial-lateral dimension, as indicated by line 2640, is 10 millimeters with an error margin of 0.5 millimeters. In a preferred embodiment, the length of an electrode on the ventral surface 2603 of the paddle body 2602, as indicated by line 2660, is 3 millimeters. In a preferred embodiment, the width of an electrode on the ventral surface 2603 of the paddle body 2602, is 2 millimeters. In a preferred embodiment, the distance between adjacent electrodes on the ventral surface 2603 of the paddle body 2602, in the rostro-caudal dimension, as indicated by line 2670, is 4 millimeters. In a preferred embodiment, the longitudinal pitch of the paddle body, as indicated by line 2675, is approximately 7 millimeters.

In a preferred embodiment, the length of an electrode on the dorsal surface 2604 of the paddle body 2602, as indicated by line 2690, is 2 millimeters. In a preferred embodiment, the width of an electrode on the dorsal surface 2603 of the paddle body 2602, as indicated by line 2680, is 3 millimeters. In a preferred embodiment, the electrodes on both the dorsal surface 2604 and the ventral surface 2603 of the paddle body 2602 have a rectangular surface area. In a preferred embodiment, the length of the ventral side electrodes, being the longest dimension of the rectangular shaped electrodes, is aligned with the rostro-caudal dimension of the paddle body 2602. In a preferred embodiment, the width of the dorsal side electrodes, being the shortest dimension of the rectangular shaped electrodes, is aligned with the rostro-caudal dimension of the paddle body 2602.

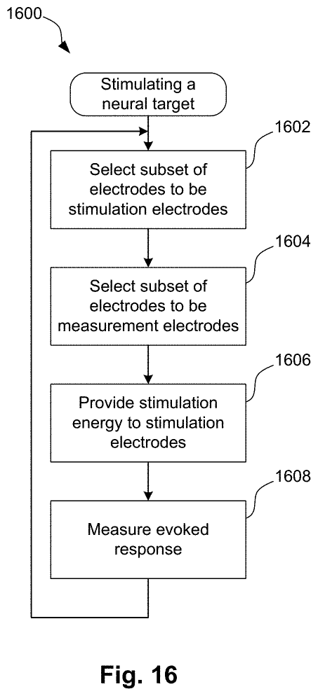

### FIG. 16—Method for Selecting Electrode Configurations

FIG. 16 is a flowchart illustrating a method for selecting electrode configurations for stimulating a neural target, according to an implementation of the present technology. In one embodiment, method 1600 is performed by electronics module 110, which comprises electrode selection module 126, stimulation circuitry 124 and measurement circuitry 128, and which is electrically coupled to each of a plurality of electrodes of a paddle electrode assembly. In one embodiment, the paddle electrode assembly comprises 24 electrodes and the electrode selection module is electrically coupled to each of the 24 electrodes.

In step 1602, the electrode selection module 126 selects a first subset of electrodes of the plurality of electrodes of the paddle electrode assembly to function as stimulation electrodes. In one embodiment, the electronics module 110 is configured to provide tripolar stimulation to a neural target in proximity to the paddle electrode assembly. The electrode selection module 126 selects three electrodes of the paddle electrode assembly as stimulation electrodes, and electrically couples the three electrodes to the stimulation circuitry 124. The stimulation electrodes may comprise two electrodes functioning as cathodes and one electrode functioning as an anode.

In step 1604, the electrode selection module 126 selects a second subset of electrodes of the plurality of electrodes of the paddle electrode assembly to function as measurement electrodes. The second subset of electrodes comprises two electrodes, wherein one electrode is configured to function as a recording electrode and one electrode is configured to function as a reference electrode. In one embodiment, the measurement circuitry 128 is configured to measure an evoked response of the neural target by measuring a potential difference between a reference electrode and a recording electrode.

In one embodiment, the electrode selection module 126 selects the second subset of electrodes based on the position of the second subset of electrodes on the paddle electrode assembly relative to the position of the first subset of electrodes on the paddle electrode assembly.

In step 1606, the electronics module 110 provides stimulation energy to the electrodes selected in step 1602. In step 1608, the electronics module 110 measures an evoked response of the neural target via the electrodes selected in step 1604.

### Electrode Selection Module Configuration

In one embodiment, the electrode selection module 126 performs a configuration step before step 1602. The configuration step provides the electrode selection module 126 with information about the performance of the electrodes of the paddle electrode assembly, which the electrode selection module 126 may use during the selection steps 1602 and 1604.

In one embodiment, the electrode selection module 126 performs the configuration step while the electronics module 110 is in-vivo, that is, while the electronics module 110 is implanted in the patient. Alternatively or additionally, the configuration step may be performed by one or more external computing devices (e.g. device 192) and the one or more external computing devices communicate configuration information to the electrode selection module 126 via the telemetry module 114.

In one embodiment, the configuration step determines information about the signal-to-artefact ratio (SAR) and signal-to-noise ratio (SNR) for various stimulus electrode configurations (SECs) and measurement electrode configurations (MECs). In one embodiment, the configuration step comprises determining SECs and MECs which exhibit SAR and SNR values with are above thresholds of acceptability.

### Shape of Electrode Surface

The paddle illustrated in FIGS. 15A and 15B comprises electrodes with a circular electrode surface. In some implementations, the shape of the electrode surface may affect one or more of the ease of manufacture of the paddle, the reliability of the paddle in use, the ease of insertion and placement of the paddle and the generation of artefacts during stimulation, including split electrode artefacts.

A surface of an electrode from which a stimulation field is project is called an electrode surface. An electrode of a paddle electrode assembly may have a circular electrode surface from which a stimulation field is projected. Alternatively, an electrode of a paddle electrode assembly may have another electrode surface shape, such as a rounded rectangle surface shape, an octagonal surface shape, a hexagonal surface shape, a pentagonal surface shape, an oblong surface shape, an oval surface shape, a rectangular surface shape or another polygonal surface shape.

Circular electrodes have 360 degree symmetry of the electrode surface plane. This symmetry can be advantageous for the assembly process as the circular symmetry of the electrode obviates the need to achieve a specific rotational alignment of the electrode. Furthermore, the symmetry and lack of corners of a circular electrode may facilitate ease of manufacture. Hexagonal electrodes provide symmetry along three axes of the surface plane, and octagonal electrodes provide symmetry along four axes of the surface plane. This symmetry may facilitate ease of assembly of the electrode in the paddle electrode assembly.

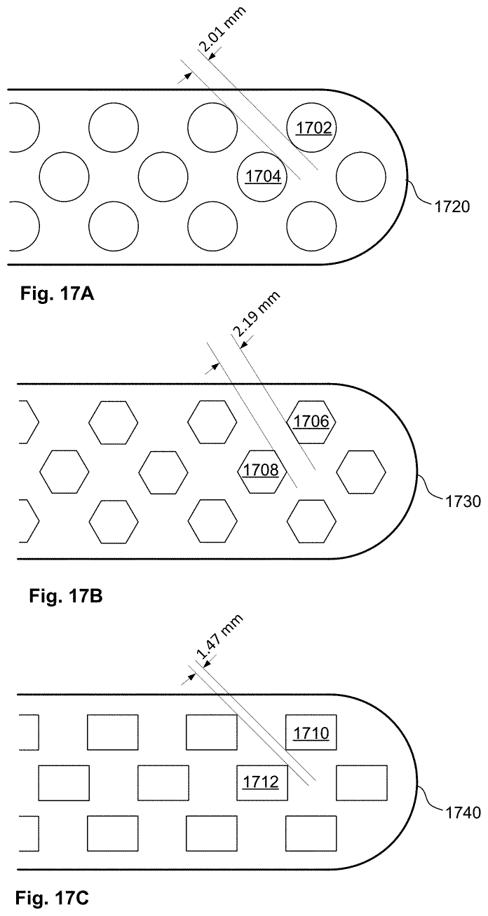

### FIG. 17—Distance Between Electrodes

The surface shape of the electrodes on the paddle can affect the distance by which the electrodes are separated on the paddle. In some implementations it may be desirable to configure the electrodes with a shape other than circular in order to increase the separation distance between neighbouring electrodes.

FIGS. 17A, 17B and 17C illustrate portions of three separate paddles, wherein each paddle has the same surface area and same number of electrodes, according to one or more implementations of the present technology. The electrodes all have an approximate surface area of 6 mm². The three paddles 1720, 1730 and 1740 differ in terms of the shape of the electrodes on each of the paddles. Paddle 1720 comprises circular electrodes arranged in a three staggered rows. The distance between the circular electrodes (e.g. 1702 and 1704) is approximately 2.01 millimetres. Paddle 1730 comprises hexagonal electrodes arranged in three staggered rows. This distance between the hexagonal electrodes (e.g. 1706 and 1708) is approximately 2.19 millimetres. Paddle 1740 comprises rectangular electrodes arranged in three staggered rows. The distance between the rectangular electrodes is approximately 1.47 millimetres.

As illustrated in FIGS. 17A to 17C, the rectangular shaped electrodes result in the smallest distance between adjacent electrodes, while the hexagonal shaped pads result in the largest distance between adjacent electrodes. Accordingly, the hexagonal electrode shape in this staggered configuration optimises for a maximum point-to-point distance between neighbouring electrodes for a given surface area of the paddle and a given surface area of the electrodes.

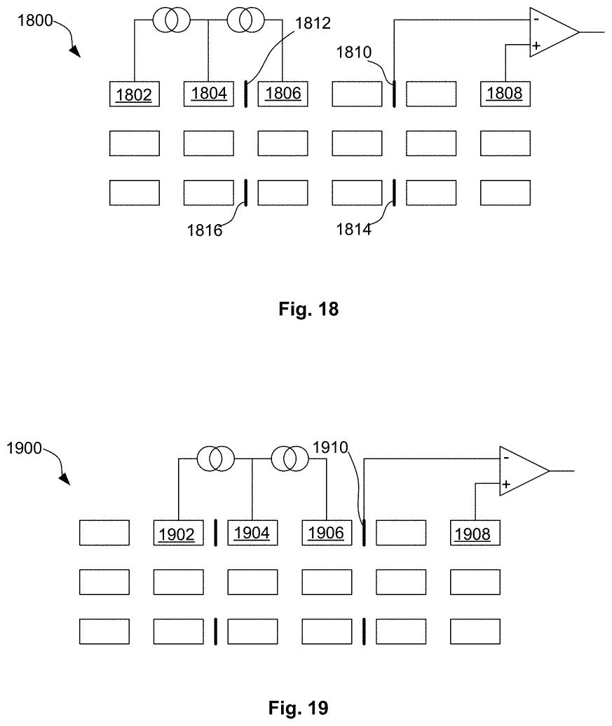

### FIGS. 18 and 19—Slim Measurement Electrodes

To ameliorate the effect of split electrode artefacts, measurement electrodes may be configured to be short in the rostro-caudal dimension. FIG. 18 illustrates an array 1800 of rectangular electrodes of a paddle electrode assembly, according to one implementation of the present technology. More particularly, the array 1800 of electrodes comprises 18 rectangular electrodes and 4 slim electrodes. The slim electrodes, 1810, 1812, 1814 and 1816, are very narrow in the rostro-caudal direction. In one implementation, the slim electrodes comprise lengths of wire. Slim electrodes that are comprised of lengths of wire may be called wire electrodes.

In the example illustrated in FIG. 18, rectangular electrodes 1802, 1804 and 1806 are configured as stimulus electrodes to provide tripolar stimulation. Slim electrode 1810 is configured as a recording electrode, with electrode 1808 configured as a reference electrode.

FIG. 19 illustrates an array 1900 of rectangular electrodes of a paddle electrode assembly, according to another implementation of the present technology. Rectangular electrodes 1902, 1904 and 1906 are configured as stimulus electrodes to provide tripolar stimulation. Slim electrode 1910 is configured as a recording electrode, with electrode 1908 configured as a reference electrode.

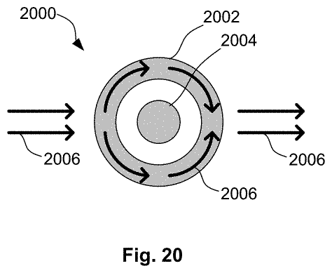

### FIG. 20—Shield Ring Electrodes

FIG. 20 illustrates a shield ring electrode 2000, according to one implementation of the present technology. A shield ring electrode may be referred to as a shielded ring electrode, a guard ring electrode, or a guard electrode. A shield ring electrode comprises an annulus electrode 2002 surrounding a centre electrode 2004. The centre electrode 2004 is configured to act as a recording electrode. The annulus electrode provides a metal ring around the recording electrode 2004. Arrows 2006 indicate current field gradients generated by a stimulation field emanating from a stimulus electrode near the shield ring electrode 2000. The annulus electrode 2002 diverts the current field gradient around the ring shape and shields the centre electrode 2004 from the current field gradient. The centre electrode 2004 may be, at least partially, shielded from charge accumulation that may result from the current field gradient. Charge accumulation may dissipate for a period that extends well into the recording period. Accordingly, the annulus electrodes ameliorates the charge accumulation that can result in a split electrode artefact affecting the ECAP measurement by the recording electrode 2004.

To ameliorate the split electrode effects that can cause a split electrode artefact, the annulus electrode may be configured, for example by the electrode selection module 126, as either a passive electrode or a stimulus electrode.

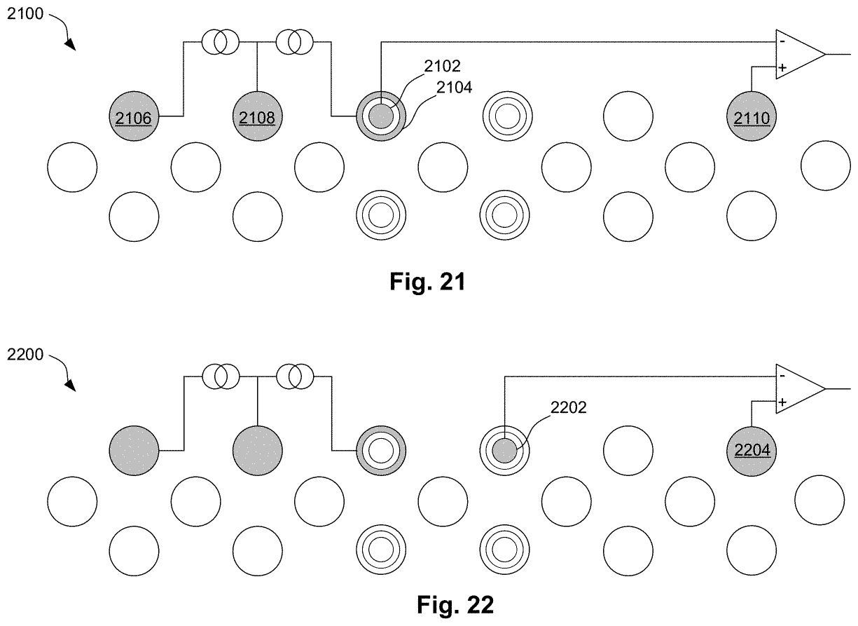

### FIGS. 21 to 25—Various SECs and MECs

FIG. 21 illustrates electrodes of a paddle electrode assembly that includes shield ring electrodes, according to one implementation of the present technology. In the example illustrated in FIG. 21, the electrode selection module has configured electrodes 2106, electrode 2108 and shield electrode 2104 as stimulus electrodes for tripolar stimulation. Centre electrode 2102 is configured as the reference electrode, and electrode 2110 is configured as the recording electrode for ECAP measurement. The annulus shape of shield electrode 2104 acts to shield the reference electrode 2102 from split electrode effects, and to ameliorate or eliminate a split electrode artefact from the ECAP measured by the measurement electrodes 2102 and 2110.

FIG. 22 illustrates electrodes of a paddle electrode assembly that includes shield ring electrodes, according to another implementation of the present technology. The configuration of shield ring electrodes and regular electrodes of paddle 2200 is the same as the configuration of shield ring electrodes and regular electrodes of paddle 2100; however, paddle 2200 has been configured with a measurement electrode configuration (MEC) that differs from the MEC of paddle 2100. In particular, centre electrode 2202 is configured as the reference electrode and electrode 2204 is configured as the recording electrode. The MEC of paddle 2200 provides increased physical separation of the reference electrode from the stimulus electrodes compared to the MEC of paddle 2100. The increased physical separation of the reference electrode from the stimulus electrode may further reduce the split electrode artefact from the ECAP measured by the measurement electrodes.

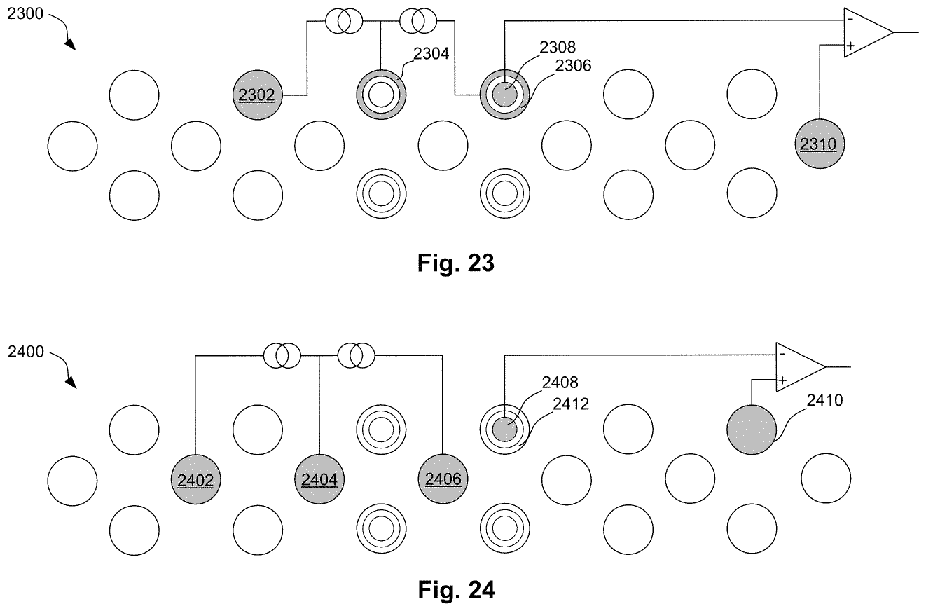

FIG. 23 illustrates electrodes of a paddle electrode assembly that includes shield ring electrodes, according to yet another implementation of the present technology. In the example illustrated in FIG. 23, the electrode selection module 126 has selected a stimulus electrode configuration (SEC) at the rostro-caudal centre of the paddle electrode assembly. More particularly, the electrode selection module has configured electrodes 2302, shield electrode 2304 and shield electrode 2306 as stimulus electrodes for tripolar stimulation. Centre electrode 2308 is configured as the reference electrode, and electrode 2310 is configured as the recording electrode for ECAP measurement. The annulus shape of shield electrode 2306 acts to shield the reference electrode 2308 from split electrode effects, and to ameliorate or eliminate a split electrode artefact from the ECAP measured by the measurement electrodes 2308 and 2310. The MEC illustrated in FIG. 23 includes a recording electrode 2310 that is offset from the stimulation electrodes (2302, 2304 and 2306) along the medial-lateral direction. Offsetting the recording electrode from the stimulation electrodes may ameliorate or eliminate a split electrode artefact from the ECAP measured by the measurement electrodes 2308 and 2310

FIG. 24 illustrates electrodes of a paddle electrode assembly that includes shield ring electrodes, according to yet another implementation of the present technology. In the example illustrated in FIG. 24, the electrode selection module 126 has selected a stimulus electrode configuration (SEC) at the medial-lateral centre of the paddle electrode assembly. More particularly, the electrode selection module has configured electrodes 2402, electrode 2404 and shield electrode 2406 as stimulus electrodes for tripolar stimulation. Centre electrode 2408 is configured as the reference electrode, and electrode 2410 is configured as the recording electrode for ECAP measurement. The annulus shape of unused shield electrode 2412 acts to shield the reference electrode 2408 from split electrode effects, and to ameliorate or eliminate a split electrode artefact from the ECAP measured by the measurement electrodes 2408 and 2410.

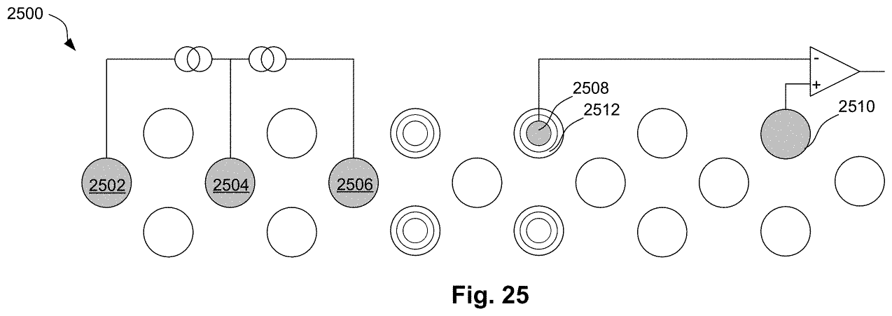

FIG. 25 illustrates electrodes of a paddle electrode assembly that includes shield ring electrodes, according to yet another implementation of the present technology. In the example illustrated in FIG. 25, the electrode selection module 126 has selected a stimulus electrode configuration (SEC) at the medial-lateral centre of the paddle electrode assembly. More particularly, the electrode selection module has configured electrodes 2502, electrode 2504 and shield electrode 2506 as stimulus electrodes for tripolar stimulation. Centre electrode 2508 is configured as the reference electrode, and electrode 2510 is configured as the recording electrode for ECAP measurement. The annulus shape of unused shield electrode 2512 acts to shield the reference electrode 2508 from split electrode effects, and to ameliorate or eliminate a split electrode artefact from the ECAP measured by the measurement electrodes 2508 and 2510.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not limiting or restrictive.

1. An implantable device for controllably stimulating a neural target, the device configured to electrically couple to a paddle electrode assembly, the paddle electrode assembly comprising a plurality of electrodes arranged on a paddle body,
the plurality of electrodes including a first group of one or more electrodes arranged on a ventral surface of the paddle body, and a second group of one or more electrodes arranged on a dorsal surface of the paddle body, and
the implantable device comprising:
stimulation circuitry, configured to provide stimulation energy to one or more electrodes of the paddle electrode assembly;
measurement circuitry, configured to measure a response evoked from the neural target by the stimulation energy and sensed by one or more electrodes of the paddle electrode assembly; and
an electrode selection module, configured to,
select at least one first electrode from the plurality of electrodes of the paddle electrode assembly and electrically couple the first electrode to the stimulation circuitry, and
select at least one second electrode from the plurality of electrodes of the paddle electrode assembly and electrically couple the second electrode to the measurement circuitry.

2. The implantable device of claim 1, wherein the second group of one or more electrodes arranged on the dorsal surface of the paddle body comprises the at least one second electrode.

3. The implantable device of claim 1, wherein the second group of one or more electrodes includes at least two electrodes.

4. The implantable device of claim 1, wherein the electrode selection module is configured to select the at least one second electrode from the plurality of electrodes of the paddle electrode assembly based on the position of the at least one second electrode relative to the at least one first electrode.

5. The implantable device of claim 1, wherein the electrode selection module is configured to select the at least one first electrode based on the position of the at least one first electrode relative to the neural target.

6. The implantable device of claim 1, wherein the electrode selection module is configured to select the at least one first electrode based on a measured response evoked from the neural target.

7. The implantable device of claim 1, wherein the at least one first electrode comprises:
a cathode electrode, configured to sink stimulus current; and
two anode electrodes, configured to source return currents,
wherein the cathode electrode is located in a position in the paddle electrode assembly between the two anode electrodes.

8. The implantable device of claim 1,
wherein the at least one second electrode comprises a recording electrode, and a reference electrode, and
wherein the measurement circuitry is configured to determine a different in potential between the recording electrode and the reference electrode.

9. The implantable device of claim 1, wherein the electrode selection module is configured to select the at least one second electrode from the plurality of electrodes of the paddle electrode assembly to maximise a distance between the at least one first electrode and the at least one second electrode.

10. A method of stimulating a neural target by an implanted paddle electrode assembly, the paddle electrode assembly comprising a plurality of electrodes arranged on a paddle body, the method comprising:
selecting, from the plurality of electrodes, a first subset of electrodes;
selecting, from the plurality of electrodes, a second subset of electrodes;
providing stimulation energy to the first subset of electrodes to stimulate the neural target; and
measuring a response of the neural target, the response evoked from the neural target by the stimulation energy, from the second subset of electrodes,
wherein the plurality of electrodes includes a first group of one or more electrodes arranged on a ventral surface of the paddle body, and a second group of one or more electrodes arranged on a dorsal surface of the paddle body.

11. The method of claim 10, wherein the second group of one or more electrodes arranged on a dorsal surface of the paddle board comprises the second subset of electrodes.

12. The method of claim 10, wherein the second group of electrodes includes at least two electrodes.

13. The method of claim 10, wherein the second subset of electrodes is selected based on the position of the second subset of electrodes relative to the first subset of electrodes.

14. The method of claim 10, wherein the first subset of electrodes is selected based on the position of the first subset of electrodes relative to the neural target.

15. The method of claim 10, further comprising selecting a second subset of electrodes to maximise a distance between any one electrode of the first subset of electrodes and any one electrode of the second subset of electrodes.

16. The method of claim 10, further comprising:
selecting, from the plurality of electrodes, a third subset of electrodes, based on the measured response of the neural target; and
providing stimulation energy to the third subset of electrodes to stimulate the neural target,
wherein the third subset of electrodes comprises at least one electrode that is not a member of the first subset of electrodes.

17. A paddle electrode assembly for an implantable medical device, the paddle electrode assembly configured to be electrically coupled to an implantable stimulation device comprising stimulation circuitry and measurement circuitry,
the paddle electrode assembly comprising,
a lead body having a proximal end and a distal end, and
a paddle disposed at the distal end of the lead body, the paddle comprising:
a paddle body, comprising a ventral surface and a dorsal surface; and
a plurality of electrodes including a first group of one or more electrodes arranged on the ventral surface of the paddle body, and a second group of one or more electrodes arranged on the dorsal surface of the paddle body,
wherein, the lead body is configured to electrically couple each electrode of the plurality of electrodes to the stimulation circuitry of the implantable stimulation device, and
wherein, the lead body is configured to electrically couple each electrode of the plurality of electrodes to the measurement circuitry of the implantable stimulation device.

18. The paddle electrode assembly of claim 17, wherein the lead body is configured to electrically couple each electrode of the second group of electrodes to the measurement circuitry of the implantable stimulation device.

19. The paddle electrode assembly of claim 17, wherein the second group of electrodes includes at least two electrodes.

20. The paddle electrode assembly of claim 17, wherein the paddle body has an elongated form defined by a rostro-caudal length and a medial-lateral width.

21. The paddle electrode assembly of claim 17, wherein the plurality of electrodes comprises a plurality of rostro-caudally aligned rows of electrodes.

22. The paddle electrode assembly of claim 17,
wherein the plurality of electrodes comprises a first rostro-caudally aligned row of electrodes and a second rostro-caudally aligned row of electrodes, and
wherein the second row of electrodes is offset from the first row of electrodes on the paddle body in a medial-lateral direction.

23. The paddle electrode assembly of claim 22, wherein the electrodes of the first rostro-caudally aligned row of electrodes are offset from the electrodes of the second rostro-caudally aligned row of electrodes in the rostro-caudal direction.

24. The paddle electrode assembly of claim 21, wherein the plurality of rostro-caudally aligned rows of electrodes comprises:
a first electrode row, positioned at a left lateral side of the paddle body;
a second electrode row, positioned at a medial midline of the paddle body; and
a third electrode row, positioned at a right lateral side of the paddle body.

25. The paddle electrode assembly of claim 24, wherein the first electrode row comprises seven electrodes, the second electrode row comprises eight electrodes and the third electrode row comprise seven electrodes.

26. The paddle electrode assembly of claim 17, wherein at least one electrode of the plurality of electrodes has an electrode surface area that is polygonal.

27. The paddle electrode assembly of claim 17, wherein at least one electrode of the plurality of electrodes has an electrode surface area that is hexagonal.

28. The paddle electrode assembly of claim 17, wherein the paddle further comprises at least one electrode having an electrode surface that is substantially shorter in the rostro-caudal direction than in the medial-lateral direction.

29. The paddle electrode assembly of claim 28, wherein the at least one electrode is configured to be a recording electrode.

30. The paddle electrode assembly of claim 28, wherein the at least one electrode is a wire electrode.

31. The paddle electrode assembly of claim 17, wherein the paddle further comprises a shield ring electrode pair, the shield ring electrode pair comprising:
a centre electrode, and
a ring electrode which at least partially encircles the centre electrode.
